SEQUENCE LISTING (11 sequences)

SEQ ID NO: 1 - protein, 5 aa, Artificial Sequence
  HCDR1 of daratumumab
SFAMS

SEQ ID NO: 2 - protein, 17 aa, Artificial Sequence
  HCDR2 of daratumumab
AISGSGGGTYYADSVKG

SEQ ID NO: 3 - protein, 13 aa, Artificial Sequence
  HCDR3 of daratumumab
DKILWFGEPVFDY

SEQ ID NO: 4 - protein, 11 aa, Artificial Sequence
  LCDR1 of daratumumab
RASQSVSSYLA

SEQ ID NO: 5 - protein, 7 aa, Artificial Sequence
  LCDR2 of daratumumab
DASNRAT

SEQ ID NO: 6 - protein, 10 aa, Artificial Sequence
  LCDR3 of daratumumab
QQRSNWPPTF

SEQ ID NO: 7 - protein, 122 aa, Artificial sequence [sequence omitted]

SEQ ID NO: 8 - protein, 107 aa, Artificial Sequence [sequence omitted]

SEQ ID NO: 9 - protein, 452 aa, Artificial Sequence
  HC of daratumumab
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  LC of daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2020/0268847 A1
(43) Pub. Date: Aug. 27, 2020

(54) Methods of Treating Newly Diagnosed Multiple Myeloma with a Combination of An Antibody that Specifically Binds CD38, Lenalidomide and Dexamethasone

(71) Applicant: Janssen Biotech, Inc., Horsham, PA (US)

(72) Inventor: Ming Qi, Phoenixville, PA (US)

(21) Appl. No.: 16/797,301

(22) Filed: Feb. 21, 2020

Related U.S. Application Data
(60) Provisional application No. 62/809,070, filed on Feb. 22, 2019.
(60) Provisional application No. 62/829,791, filed on Apr. 5, 2019.
(60) Provisional application No. 62/829,804, filed on Apr. 5, 2019.
(60) Provisional application No. 62/829,814, filed on Apr. 5, 2019.

(51) Int. Cl. A61K 38/17; A61K 31/573; A61K 31/454; A61P 35/00

(52) U.S. Cl. CPC A61K 38/1774; A61K 31/573; A61K 9/0019; A61P 35/00; A61K 31/454

(57) ABSTRACT

Disclosed herein are methods of treating multiple myeloma using an antibody that specifically binds CD38 in combination with lenalidomide and dexamethasone.

### CROSS-REFERENCE TO RELATED APPLICATIONS

This application claims the benefit of U.S. Provisional Application Ser. No. 62/829,814, filed 5 Apr. 2019, U.S. Provisional Application Ser. No. 62/829,804, filed 5 Apr. 2019, U.S. Provisional Application Ser. No. 62/829,791, filed 5 Apr. 2019 and U.S. Provisional Application Ser. No. 62/809,070, filed 22 Feb. 2019, the entire contents of which are incorporated herein by reference.

### FIELD OF THE INVENTION

Disclosed herein are methods of treating multiple myeloma using an antibody that specifically binds CD38 in combination with lenalidomide and dexamethasone.

### SEQUENCE LISTING

This application contains a Sequence Listing submitted via EFS-Web, the entire content of which is incorporated herein by reference. The ASCII text file, created on 20 Feb. 2020, is named JBII6048USNP1ST25.txt and is 13 kilobytes in size.

### BACKGROUND OF THE INVENTION

Multiple myeloma is a malignant disorder of the plasma cells, characterized by uncontrolled and progressive proliferation of a plasma cell clone. The disease leads to progressive morbidity and eventual mortality by lowering resistance to infection and causing significant skeletal destruction (with bone pain, pathological fractures, and hypercalcemia), anaemia, renal failure, neurological complications and hyperviscosity syndrome.

Multiple myeloma remains incurable with standard chemotherapy, despite the availability of multi-agent therapies.

There remains a need for new therapeutic options for the frontline setting that can better control the disease and provide deeper, more sustained responses and better long-term outcomes, including maintenance of health-related quality of life.

### SUMMARY OF THE INVENTION

The disclosure provides a method of treating a subject with newly diagnosed multiple myeloma, comprising administering to the subject a combination therapy comprising daratumumab, lenalidomide and dexamethasone, wherein the method achieves an improved clinical efficacy endpoint when compared to a clinical efficacy endpoint achieved if the subject were administered a combination of lenalidomide and dexamethasone.

The disclosure also provides a method of treating a subject with newly diagnosed multiple myeloma who is ineligible for high dose chemotherapy (HDC) and autologous stem cell transplant (ASCT), comprising administering or providing for administration to the subject daratumumab, wherein daratumumab is administered as a combination therapy with lenalidomide and dexamethasone, and wherein the method achieves an improved clinical efficacy endpoint when compared to a clinical efficacy endpoint achieved if the subject were administered a combination of lenalidomide and dexamethasone.

The disclosure also provides a method of treating a subject with newly diagnosed multiple myeloma, comprising administering to the subject a combination therapy demonstrated to increase a likelihood of achieving a very good partial response (VGPR) or better in subjects with multiple myeloma, wherein the combination therapy comprises daratumumab, lenalidomide and dexamethasone.

The disclosure also provides a method of treating a subject with newly diagnosed multiple myeloma, comprising administering to the subject a combination therapy demonstrated to increase a likelihood of achieving a negative status for minimal residual disease (MRD) in subjects with newly diagnosed multiple myeloma, wherein the combination therapy comprises daratumumab, lenalidomide and dexamethasone.

The disclosure also provides a method of treating a subject with newly diagnosed multiple myeloma, comprising administering to the subject a combination therapy demonstrated to increase a likelihood of achieving a complete response (CR) or better in subjects with newly diagnosed multiple myeloma, wherein the combination therapy comprises daratumumab, lenalidomide and dexamethasone.

The disclosure also provides a method of treating a subject with newly diagnosed multiple myeloma, comprising administering to the subject a combination therapy demonstrated to reduce a risk of progression of multiple myeloma or death in subjects with newly diagnosed multiple myeloma, wherein the combination therapy comprises daratumumab, lenalidomide and dexamethasone.

The disclosure also provides a method of treating a subject with newly diagnosed multiple myeloma, comprising:

providing a healthcare professional (HCP) daratumumab;
providing the HCP information that treating the subject with a combination therapy comprising daratumumab, lenalidomide and dexamethasone achieves an improved clinical efficacy endpoint when compared to a clinical efficacy endpoint achieved if the subject were treated with a combination of lenalidomide and dexamethasone; wherein performing the steps a) and b) results in the subject with newly diagnosed multiple myeloma to receive the combination therapy comprising daratumumab, lenalidomide and dexamethasone by the HCP or by self-administration as instructed by the HCP, thereby treating the subject having the newly diagnosed multiple myeloma.

The disclosure also provides a method of providing daratumumab to a HCP for the HCP to treat a subject with newly diagnosed multiple myeloma with a combination therapy comprising daratumumab, lenalidomide and dexamethasone, wherein the treatment with the combination therapy comprising daratumumab, lenalidomide and dexamethasone achieves an improved clinical efficacy endpoint when compared to a clinical efficacy endpoint achieved if the subject were treated with a combination of lenalidomide and dexamethasone, comprising:

manufacturing daratumumab;
providing the HCP information that treatment with the combination therapy comprising daratumumab, lenalidomide and dexamethasone achieves the improved clinical efficacy endpoint; and shipping daratumumab to the HCP or to an authorized distributor of daratumumab for the HCP to purchase daratumumab; thereby providing daratumumab to the HCP.

The disclosure also provides a method of providing a treatment option for a HCP to treat a subject with newly diagnosed multiple myeloma with a combination therapy comprising daratumumab, lenalidomide and dexamethasone, wherein the treatment with the combination therapy comprising daratumumab, lenalidomide and dexamethasone achieves an improved clinical efficacy endpoint when compared to a clinical efficacy endpoint achieved if the subject were treated with a combination of lenalidomide and dexamethasone, comprising:

manufacturing daratumumab;
providing the HCP information that the combination therapy comprising daratumumab, lenalidomide and dexamethasone achieves the improved clinical efficacy endpoint; and shipping daratumumab to the HCP or to an authorized distributor of daratumumab for the HCP to purchase daratumumab, thereby providing the treatment option for the HCP.

### BRIEF DESCRIPTION OF THE DRAWINGS

The summary, as well as the following detailed description, is further understood when read in conjunction with the appended drawings. For the purpose of illustrating the disclosed methods, the drawings show exemplary embodiments of the methods; however, the methods are not limited to the specific embodiments disclosed. In the drawings:

FIG. 1 shows the results of the Kaplan-Meier estimates of progression-free survival among patients in the intention-to-treat population. The daratumumab (DARZALEX®) group received treatment with daratumumab (DARZALEX®), lenalidomide, and dexamethasone; the control group received treatment with lenalidomide and dexamethasone. The interim analysis of progression-free survival was performed after 240 events of disease progression or death had occurred (62% of planned 390 events for the final analysis).

FIG. 2 shows the results of an analysis of progression-free survival in prespecified subgroups in the intention-to-treat population. The daratumumab (DARZALEX®) group received treatment with daratumumab)(DARZALEX®, lenalidomide, and dexamethasone; the control group received treatment with lenalidomide and dexamethasone. The International Staging System (ISS) disease stage is derived based on the combination of serum β2-microglobulin and albumin levels, with higher stages indicating more advanced disease. Impaired baseline hepatic function included mild impairment (total bilirubin level ≤the upper limit of the normal range (ULN) and aspartate aminotransferase level >the ULN, or total bilirubin level >the ULN and ≤1.5 times the ULN), moderate impairment (total bilirubin level >1.5 times and ≤3 times the ULN), and severe impairment (total bilirubin level >3 times the ULN). The subgroup analysis for the type of myeloma was performed on data from patients who had measurable disease in serum or urine. A high-risk cytogenetic profile was defined by the detection of a del17p, t(14;16), and/or t(4;14) cytogenetic abnormality on fluorescence in situ hybridization testing or karyotype. Eastern Cooperative Oncology Group (ECOG) performance status was scored on a scale from 0 to 5, with 0 indicating no symptoms and higher scores indicating increasing disability. NE denotes not estimable.

### DETAILED DESCRIPTION OF THE INVENTION

All publications, including but not limited to patents and patent applications, cited in this specification are herein incorporated by reference as though fully set forth.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.” “About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“About once a week” refers to an approximate number, and can include every 7 days±two days, i.e., every 5 days to every 9 days. The dosing frequency of “once a week” thus can be every five days, every six days, every seven days, every eight days, or every nine days.

“About once in two weeks” refers to an approximate number, and can include every 14 days±two days, i.e., every 12 days to every 16 days.

“About once in three weeks” refers to an approximate number, and can include every 21 days±two days, i.e., every 19 to every 23 days.

“About once in four weeks” refers to an approximate number, and can include every 28 days±two days, i.e., every 26 to every 30 days.

“About once in five weeks” refers to an approximate number, and can include every 35 days±two days, i.e., every 33 to every 37 days.

“About once in six weeks” refers to an approximate number, and can include every 42 days±two days, i.e., every 40 to every 38 days.

“About twice a week” refers to an approximate number, can include twice in one week, e.g., a first dose on day 1 and a second dose on day 2, day 3, day 4, day 5, day 6 or day 7 of the week, the first dose on day 2 and the second dose on day 3, day 4, day 5, day 6 or day 7 of the week, the first dose on day 3 and the second dose on day 4, day 5, day 6 or day 7 of the week, the first dose on day 4 and the second dose on day 5, day 6 or day 7 of the week, the first dose on day 5 and the second dose on day 6 or day 7 of the week, the first dose on day 6 and the second dose on day 7 of the week.

“Adverse event” (AE) refers to any untoward medical occurrence in a clinical study subject administered an antibody that specifically binds CD38, such as daratumumab. An AE does not necessarily have a causal relationship with the treatment. An AE can therefore be any unfavorable and unintended sign (including an abnormal finding), symptom, or disease temporally associated with the use of a medicinal (investigational or non-investigational) product, whether or not related to the antibody that specifically binds CD38, such as daratumumab.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.” “Antibody” includes immunoglobulin molecules belonging to any class, IgA, IgD, IgE, IgG and IgM, or sub-class IgA1, IgA2, IgG1, IgG2, IgG3 and IgG4 and including either kappa (κ) and lambda (2) light chain. Antibodies include monoclonal antibodies including human, humanized and chimeric monoclonal antibodies. Full-length antibody molecules are comprised of two heavy chains (HC) and two light chains (LC) inter-connected by disulfide bonds. Each heavy chain is comprised of a heavy chain variable region (VH) and a heavy chain constant region (comprised of domains CHL hinge, CH2 and CH3). Each light chain is comprised of a light chain variable region (VL) and a light chain constant region (CL). The VH and the VL regions may be further subdivided into regions of hypervariability, termed complementarity determining regions (CDRs), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Biosimilar” (of an approved reference product/biological drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Cancer” refers to an abnormal growth of cells which tend to proliferate in an uncontrolled way and, in some cases, to metastasize (spread) to other areas of a patient's body.

“CD38” refers to human cluster of differentiation 38 protein, a glycoprotein expressed on immune cells, including plasma cells, natural killer cells and sub-populations of B and T cells.

“Clinical efficacy endpoint” or “clinical endpoint” refers to an outcome that represents a clinical benefit, such as progression-free survival (PFS), time to disease progression (TTP), time to next treatment, overall response rate (ORR), proportion of subjects achieving partial response (PR), proportion of subjects achieving very good partial response (VGPR), proportion of subjects achieving complete response (CR), proportion of subjects achieving stringent complete response (sCR), proportion of subjects achieving a negative status for minimal residual disease (MRD), or proportion of subjects achieving both sCR and negative status for MRD.

“Clinically proven” refers to clinical efficacy results that are sufficient to meet approval standards of U.S. Food and Drug Administration (FDA), European Medicines Agency (EMA) or a corresponding national regulatory agency. For example, the clinical study may be an adequately sized, randomized, double-blinded controlled study used to clinically prove the effects of the drug.

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Combination” refers to a combination of two or more therapeutics or drugs that can be administered either together or separately.

“Complementarity determining regions” (CDRs) are “antigen binding sites” in an antibody. CDRs may be defined based on sequence variability (Wu and Kabat, J Exp Med 132:211-250, 1970; Kabat et al., Sequences of Proteins of Immunological Interest, 5th Ed. Public Health Service, National Institutes of Health, Bethesda, Md., 1991) or based on alternative delineations (see Lefranc et al., Dev Comparat Immunol 27:55-77, 2003). The International ImMunoGeneTics (IMGT) database (http//www_imgtorg) provides a standardized numbering and definition of antigen-binding sites.

“Complete response rate or better” (CR rate or better) refers to the proportion of subjects achieving CR or stringent complete response (sCR) during or after the treatment.

“Comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristics” of the claimed invention. Embodiments described in terms of the phrase “comprising” (or its equivalents) also provide as embodiments those independently described in terms of “consisting of” and “consisting essentially of.”

“Corticosteroid” refers to a class of steroid hormones that are produced in the adrenal cortex or produced synthetically refers to dexamethasone, methylprednisolone, prednisolone and prednisone. Dexamethasone is marketed under the trade name DECARON®_{.}

“Cycle” refers to the administration schedule of one or more therapeutics or drugs and refers to the period of time when the one or more therapeutics or drugs is administered to a subject. Cycle may include days in which the drug is administered and periods of rest in which the drug is not administered. Cycle length may vary, and can be for example 2 weeks, 3 weeks, 28-days (or 4 weeks), 5 weeks or 6 weeks.

“Daily” in the context of dosing refers to a total dose of a drug such as lenalidomide administered to a subject in a day. The dose may be divided to two or more administrations during the day, or given as one administration per day. For example, the total dose may be 25 mg daily administered as a singe dose.

“Daratumumab” refers to an antibody that specifically binds CD38 comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5, a LCDR3 of SEQ ID NO: 6, a heavy chain variable region (VH) of SEQ ID NO: 7, a light chain variable region (VL) of SEQ ID NO: 8, a heavy chain (HC) of SEQ ID NO: 9 and a light chain (LC) of SEQ ID NO: 10. Daratumumab is marketed under the trade name DARZALEX®. “Daratumumab” refers to any drug comprising daratumumab as an active ingredient, including biosimilars of DARZALEX®.

“Daratumumab-containing drug product” refers to any drug product in which daratumumab is an active ingredient.

“Dexamethasone” is designated chemically as 9-fluoro-11β,17,21-trihydroxy-16α-methylpregna-1,4-diene-3,20-dione. The structure of dexamethasone is shown in Formula 1

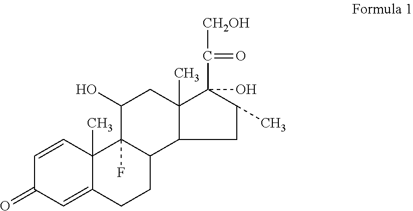

“Dose” refers to the amount or quantity of the therapeutic or the drug to be taken each time.

“Dosage” refers to the information of the amount of the therapeutic or the drug to be taken by the subject and the frequency of the number of times the therapeutic is to be taken by the subject.

“Drug product” (DP) refers to a finished dosage form, for example, a tablet, capsule or solution that contains an active pharmaceutical ingredient (e.g., drug substance), generally, but not necessarily, in association with inactive ingredients.

“Drug substance” (DS) refers to any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure or function of the body.

“Duration of response” refers to the time between the date of initial documentation of a response (partial response (PR) or better) to the date of the first documented evidence of progressive disease.

“Effective” refers to a dose or dosage of a therapeutic or a drug (such as an antibody that specifically binds CD38 such as daratumumab) or a combination of therapeutics or drugs that provides a therapeutic effect for a given condition and administration regimen in a subject receiving or who has received the therapeutic or the drug or the combination of the therapeutics or drugs. “Effective” is intended to mean an amount sufficient to reduce and/or prevent a clinically significant deficit in the activity, function and response of the subject, or to cause an improvement in a clinically significant condition in the subject.

“Frontline” or “firstline” therapy refers to the first treatment of a disease, such as multiple myeloma, administered to the subject.

“Glutamic acid derivative” refers to immunomodulatory drugs that are derivatives of glutamic acid such as lenalidomide, thalidomide and pomalidomide. Lenalinomide is marketed under the trade name REVLIMID®. Thalidomide is marketed under the trade name THALOMID®. Pomalidomide is marketed under the trade name POMALYST®

“Healthcare professional” (HCP) refers to a medical doctor, a nurse, a nurse's assistant, or a person working under direct instructions by the medical doctor or the nurse, or any person working in a hospital or a place in which treatment can be provided to the subject.

“High dose chemotherapy” (HDC) and “autologous stem cell transplant” (ASCT) refer to the treatment of subjects with newly diagnosed multiple myeloma who are considered fit. Subjects under the age of 65 years who have one or more comorbidities likely to have a negative impact on tolerability of HDC and ASCT or subjects over the age of 65 years are usually not considered eligible for HDC and ASCT due to their frail physical status which increase the risk of mortality and transplant-related complications (e.g. subjects are “ineligible”). An exemplary comorbidity is a renal dysfunction. Exemplary HDC regimens are melphalan at a dose of 200 mg/m² with dose reductions based on age and renal function, cyclophosphamide and melphalan, carmustine, etoposide, cytarabine, and melphalan (BEAM), high-dose idarubicin, cyclophosphamide, thiotepa, busulfan, and cyclophosphamide, busulfan and melphalan, and high-dose lenalidomide (Mahajan et al., Ther Adv Hematol 9:123-133, 2018).

“High risk multiple myeloma” refers to multiple myeloma that is characterized by one or more cytogenetic abnormalities del17p, t(4;14), t(14;20), t(14;16) or del13, or any combination thereof.

“Information” refers to reported results from clinical trials and can be provided in written or electronic form, or orally, or it can be available on internet.

“Infusion related reaction” (IRR) refers to any sign or symptom experienced by a subject during the administration of a drug or a therapeutic or any event occurring within 24-hours of administration. IRRs are typically classified as Grade 1, 2, 3 or 4.

“Label” and “labeling” are used interchangeably herein and refers to all labels and displays of written, printed, or graphic information on, in or accompanying a container or package comprising a drug, such as daratumumab, or otherwise available electronically or on internet. “Label” and “labeling” include package insert and prescribing information.

“Lenalidomide” a thalidomide analogue, is an immunomodulatory agent with antiangiogenic and antineoplastic properties. The chemical name is 3-(4-amino-1-oxo 1,3-dihydro-2H-isoindol-2-yl) piperidine-2,6-dione and it has the structure shown in Formula 2. Lenalinomide is marketed under the trade name REVLIMID®.

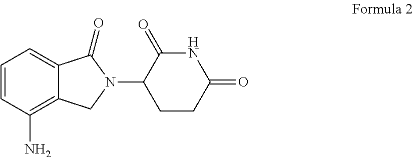 [SMILES: Nc1cccc2c1CN(C1CCC(=O)NC1=O)C2=O]

“Minimal residual disease” (MRD) refers to a small number of clonal multiple myeloma cells that remain in the patient after treatment and/or during remission.

“MRD negative” or “negative status for MRD” refers to a ratio of 1:10×10⁵ or less clonal multiple myeloma cells in a bone marrow aspirate sample obtained from the subject.

“MRD negativity rate” refers to the proportion of subjects assessed as MRD negative at any timepoint after the date of randomization.

“Multiple myeloma” refers to a malignant disorder of plasma cells characterized by uncontrolled and progressive proliferation of one or more malignant plasma cells. The abnormal proliferation of plasma (myeloma) cells causes displacement of the normal bone marrow leading to dysfunction in hematopoietic tissue and destruction of the bone marrow architecture, resulting in progressive morbidity and eventual mortality.

“Newly diagnosed” refers to a human subject who has been diagnosed with but has not yet received treatment for multiple myeloma.

“Overall response rate” (ORR) refers to the proportion of subjects who achieve partial response (PR), very good partial response (VGPR), complete response (CR) or stringent complete response (sCR) during or after the treatment.

“Overall survival” (OS) is defined as the time from initiation of therapy to the date of death due to any cause. For the purpose of the clinical trial described in the example, OS is defined as the time from randomization of study population to the date of the patient's death.

“Percent w/v” (% w/v) refers to weight in grams per 100 m.

“Per week” refers to a total dose of a drug such as dexamethasone administered to a subject in one week. The dose may be divided to two or more administrations during the same day or different days. For example, the total dose may be 40 mg administered 20 mg on day 1 and 20 mg on day 3 of a week.

“Pharmaceutically acceptable carrier” or “excipient” refers to an ingredient in a pharmaceutical composition, other than the active ingredient, which is nontoxic to a subject. A pharmaceutically acceptable carrier includes, but is not limited to, a buffer, stabilizer or preservative.

“Progression-free survival” (PFS) means time from initiation of therapy to first evidence of disease progression or death due to any cause, whichever occurs first. For the purpose of the clinical trial described in the example, PFS is defined as the duration from the date of randomization of study population to the first documented progressive disease or death due to any cause, whichever occurs first.

“Progression-free survival with the first subsequent therapy” (PFS2) is defined as the time from initiation of therapy to progression on the next line of therapy or death, whichever comes first

“Progressive disease” (PD), “stable disease” (SD), “partial response” (PR), “very good partial response” (VGPR), “complete response” (CR) and “stringent complete response” (sCR) refer to response to treatment and take their customary meanings as will be understood by a person skilled in the art of designing, conducting, or reviewing clinical trials. Response to treatment may be assessed using International Myeloma Working Group (IMWG) uniform response criteria recommendations (International Uniform Response Criteria Consensus Recommendations) as shown in Table 1.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Reference product” refers to an approved biological product such as DARZALEX® brand of daratumumab against which a biosimilar product is compared. A reference product is approved in the U.S. based on, among other things, a full complement of safety and effectiveness data.

“Safe” as it relates to a composition, dose, dosage regimen, treatment or method with a therapeutic or a drug (such as an antibody that specifically binds CD38, for example daratumumab) refers to a favorable benefit:risk ratio with an acceptable frequency and/or acceptable severity of adverse events (AEs) and/or treatment-emergent adverse events (TEAEs) compared to the standard of care (such as for example a combination of lenalidomide and dexamethasone) or to another comparator.

“Safe and effective” refers to an amount and/or dosage of a drug (such as an antibody that specifically binds CD38, for example daratumumab) or a combination of drugs that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is evaluated in laboratory, animal and human clinical testing to determine the highest tolerable dose or the optimal dose of the drug or the combination of drugs needed to achieve the desired benefit. Efficacy is evaluated in human clinical trials and determining whether the drug or the combination of drugs demonstrates a health benefit over a placebo or other intervention. Safe and effective drugs or a combination of drugs are granted marketing approval by the FDA for their indicated use.

An antibody that “specifically binds CD38” refers to antibody binding CD38 with greater affinity than to other antigens. Typically, the antibody binds to CD38 with an equilibrium dissociation constant (K_{D}) of about 1×10⁻⁸ M or less, for example about 1×10⁻⁹ M or less, about 1×10⁻¹⁰ M or less, about 1×10⁻¹¹M or less, or about 1×10¹²M or less, typically with a K_{D} that is at least one hundred-fold less than its K_{D} for binding to a non-specific antigen (e.g., BSA, casein). The K_{D} may be measured using standard procedures. Antibodies that specifically bind CD38 may, however, have cross-reactivity to other related antigens, for example to the same antigen from other species (homologs), such as monkey, for example Macaca fascicularis (cynomolgus, cyno), Pan troglodytes (chimpanzee, chimp) or Callithrix jacchus (common marmoset, marmoset).

“Subject” refers to a human patient. The terms “subject” and “patient” can be used interchangeably herein.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

“Time to disease progression” (TTP) means time from the date of randomization to the date of first documented evidence of progressive disease.

“Time to next treatment” refers to the time from randomization to the start of the next-line treatment.

“Time to response” refers to the time between the randomization and the first efficacy evaluation that the subject has met all criteria for PR or better.

“Time to subsequent anti-myeloma therapy” refers to the time from the initiation of therapy to documentation of administration of a new anti-myeloma therapy to the subject.

“Treat”, “treating” or “treatment” refers to therapeutic treatment. Individuals in need of treatment include those subjects diagnosed with the disorder of a symptom of the disorder. Subject that may be treated also include those prone or susceptible to have the disorder, or those in which the disorder is to be prevented. Beneficial or desired clinical results include alleviation of symptoms, diminishment of extent of disease, stabilized (i.e., not worsening) state of disease, delay or slowing of disease progression, amelioration or palliation of the disease state, disease remission (whether partial or total) and prolonging survival as compared to expected survival if a subject was not receiving treatment or was receiving another treatment.

“Treatment emergent adverse events” (TEAE) as used herein takes its customary meaning as will be understood by a person skilled in the art of designing, conducting, or reviewing clinical trials and refers to an AE considered associated with the use of an antibody that specifically binds CD38, for example daratumumab, if the attribution is possible, probable, or very likely.

“Unacceptable adverse events” and “unacceptable adverse reaction” refers to all harm or undesired outcomes associated with or caused by administration of a pharmaceutical composition or a therapeutic, and the harm or undesired outcome reaches such a level of severity that a regulatory agency deems the pharmaceutical composition or the therapeutic unacceptable for the proposed use.

“Very good partial response or better” (VGPR rate or better) refers to the proportion of subjects achieving VGPR, complete response (CR) or stringent complete response (sCR) during or after the treatment.

### Multiple Myeloma

Multiple myeloma causes significant morbidity and mortality. It accounts for approximately 1% of all malignancies and 13% of hematologic cancers worldwide. Approximately 50,000 patients per year are diagnosed with multiple myeloma in the EU and US, and 30,000 patients per year die due to multiple myeloma.

The majority of patients with multiple myeloma produce a monoclonal protein (paraprotein, M-protein or M-component) which is an immunoglobulin (Ig) or a fragment of one that has lost its function (Kyle and Rajkumar, Leukemia 23:3-9, 2009; Palumbo and Anderson, N Engl J Med 364:1046-1060, 2011). Normal immunoglobulin levels are compromised in patients, leading to susceptibility of infections. The proliferating multiple myeloma cells displace the normal bone marrow leading to dysfunction in normal hematopoietic tissue and destruction of the normal bone marrow architecture, which is reflected by clinical findings such as anemia, paraprotein in serum or urine, and bone resorption seen as diffuse osteoporosis or lytic lesions shown in radiographs (Kyle et al., Mayo Clin Proc 78:21-33, 2003). Furthermore, hypercalcemia, renal insufficiency or failure, and neurological complications are frequently seen. A small minority of patients with multiple myeloma are non-secretory.

Treatment choices for multiple myeloma vary with age, comorbidity, the aggressiveness of the disease, and related prognostic factors (Palumbo and Anderson, N Engl J Med 364:1046-1060, 2011). Newly diagnosed patients with multiple myeloma are typically categorized into 2 subpopulations usually defined by their age and suitability for the subsequent approach to treatment. Younger patients will typically receive an induction regimen followed by consolidation treatment with high-dose chemotherapy (HDC) and autologous stem cell transplantation (ASCT). For those not considered suitable for HDC and ASCT, longer-term treatment with multi-agent combinations including alkylators, high-dose steroids, and novel agents are currently considered as standards of care. In general, patients over the age of 65 or with significant comorbidities are usually not considered eligible for HDC and ASCT. For many years, the oral combination melphalan-prednisone (MP) was considered the standard of care for patients with multiple myeloma who were not eligible for ASCT (Gay and Palumbo, Blood Reviews 25:65-73, 2011). The advent of immunomodulatory agents (IMiDs) and proteasome inhibitors (PIs) has led to a multiplicity of new treatment options for newly diagnosed patients not considered suitable for transplant-based therapy.

### Multiple Myeloma Diagnosis

Subjects afflicted with multiple myeloma satisfy the CRAB (calcium elevation, renal insufficiency, anemia and bone abnormalities) criteria, and have clonal bone marrow plasma cells ≥10% or biopsy-proven bony or extramedullary plasmacytoma, and measurable disease. Measurable disease is defined by any of the following;
IgG myeloma: Serum monoclonal paraprotein (M-protein) level ≥1.0 g/dL or urine M-protein level ≥200 mg/24 hours; or
IgA, IgM, IgD, or IgE multiple myeloma: serum M-protein level ≥0.5 g/dL or urine M-protein level ≥200 mg/24 hours; or
Light chain multiple myeloma without measurable disease in serum or urine: Serum immunoglobulin free light chain ≥10 mg/dL and abnormal serum immunoglobulin kappa lambda free light chain ratio.

### CRAB Criteria

Hypercalcemia: serum calcium >0.25 mM/L (>1 mg/dL) higher than the upper limit of the normal range [ULN] or >2.75 mM/L (>11 mg/dL)
Renal insufficiency: creatinine clearance <40 mL/min or serum creatinine >177 μM/L (>2 mg/dL)
Anemia: hemoglobin >2 g/dL below the lower limit of normal or hemoglobin <10 g/dL
Bone lesions: one or more osteolytic lesions on skeletal radiography, CT, or PET-CT

Response to treatment may be assessed using International Myeloma Working Group (IMWG) uniform response criteria recommendations (International Uniform Response Criteria Consensus Recommendations) as shown in Table 1.

TABLE 1

| Response | Response Criteria |
|---|---|
| Stringent | CR as defined below, plus |
| complete | Normal FLC ratio, and |
| Response | Absence of clonal PCs by immunohistochemistry, |
| (sCR) | immunofluorescence or 2-to 4-color flow cytometry |
| Complete | Negative immunofixation on the serum and urine, and |
| response | Disappearance of any soft tissue plasmacytomas, and |
| (CR) | <5% PCs in bone marrow |
| Very good | Serum and urine M-component detectable by |
| partial | immunofixation but not on electrophoresis, or |
| Response | ≥90% reduction in serum M-protein |
| (VGPR) | plus urine M-protein <100 mg/24 hours |
| Partial | ≥50% reduction of serum M-protein and reduction in |
| response | 24-hour urinary M-protein by ≥90% or to <200 mg/24 hours |
| (PR) | If the serum and urine M-protein are not measurable, |
| | a decrease of ≥50% in the difference between involved |
| | and uninvolved FLC levels is required in place |
| | of the M-protein criteria |
| | If serum and urine M-protein are not measurable, |
| | and serum free light assay is also not measurable, ≥50% |
| | reduction in bone marrow PCs is required in place of |
| | M-protein, provided baseline bone marrow plasma |
| | cell percentage was ≥30% |
| | In addition to the above criteria, if present at baseline, |
| | a ≥50% reduction in the size of soft tissue plasmacytomas |
| | is also required. |
| Stable | Not meeting criteria for CR, VGPR, PR, or progressive |
| disease | disease |
| (SD) | |
| Progressive | Increase of 25% from lowest response value in any |
| disease | one of the following: |
| (PD) | Serum M-component (absolute increase must be ≥0.5 g/dL), |
| | Urine M-component (absolute increase must be |
| | ≥200 mg/24 hours), |
| | Only in subjects without measurable serum and |
| | urine M-protein levels: the difference between |
| | involved and uninvolved FLC levels (absolute |
| | increase must be >10 mg/dL) |
| | Only in subjects without measurable serum and urine |
| | M-protein levels and without measurable disease |
| | by FLC levels, bone marrow PC percentage (absolute |
| | percentage must be ≥10%) |
| | Bone marrow plasma cell percentage: the absolute |
| | percentage must be >10% |
| | Definite development of new bone lesions or soft tissue |
| | plasmacytomas or definite increase in the size of |
| | existing bone lesions or soft tissue plasmacytomas |
| | Development of hypercalcemia (corrected serum calcium |
| | >11.5 mg/dL) that can be attributed solely to the PC |
| | proliferative disorder |

EBMT = European Group for Blood and Marrow Transplantation; FLC = free light chain; PC = plasma cell

### Methods of the Disclosure

The disclosure provides a method of treating a subject with multiple myeloma, comprising administering to the subject a combination therapy comprising daratumumab and one or more immunomodulatory agents or bortezomib.

In some embodiments, the one or more immunomodulatory agents is a glutamic acid derivative.

In some embodiments, the glutamic acid derivative is lenalidomide or pomalidomide.

In some embodiments, multiple myeloma is relapsed multiple myeloma.

In some embodiments, multiple myeloma is refractory multiple myeloma.

In some embodiments, multiple myeloma is newly diagnosed multiple myeloma.

The disclosure also provides a method of treating a subject with newly diagnosed multiple myeloma, comprising administering to the subject a combination therapy comprising daratumumab, lenalidomide and dexamethasone.

The disclosure also provides a method of treating a subject with newly diagnosed multiple myeloma, comprising administering to the subject a combination therapy comprising daratumumab, lenalidomide and dexamethasone, wherein the method achieves an improved clinical efficacy endpoint when compared to a clinical efficacy endpoint achieved if the subject were administered a combination of lenalidomide and dexamethasone.

The disclosure also provides method of treating a subject with newly diagnosed multiple myeloma who is ineligible for high dose chemotherapy (HDC) and autologous stem cell transplant (ASCT), comprising administering or providing for administration to the subject daratumumab, wherein daratumumab is administered as a combination therapy with lenalidomide and dexamethasone, and wherein the method achieves an improved clinical efficacy endpoint when compared to a clinical efficacy endpoint achieved if the subject were administered a combination of lenalidomide and dexamethasone.

In some embodiments, the improved clinical efficacy endpoint is an increased likelihood of achieving a complete response (CR) or better, an increased likelihood of achieving a very good partial response (VGPR) or better, an increased likelihood of achieving a negative status for minimal residual disease (MRD), a reduced risk of progression of multiple myeloma or death, a prolonged progression-free survival (PFS), or an increased likelihood of achieving a 30-month rate of progression-free survival.

The disclosure also provides a method of treating a subject with newly diagnosed multiple myeloma, comprising administering to the subject a combination therapy demonstrated to increase a likelihood of achieving a VGPR or better in subjects with multiple myeloma, wherein the combination therapy comprises daratumumab, lenalidomide and dexamethasone.

In some embodiments, the likelihood of achieving the VGPR or better is about 79% or higher.

The disclosure also provides a method of treating a subject with newly diagnosed multiple myeloma, comprising administering to the subject a combination therapy demonstrated to increase a likelihood of achieving a negative status for MRD in subjects with newly diagnosed multiple myeloma, wherein the combination therapy comprises daratumumab, lenalidomide and dexamethasone.

In some embodiments, the likelihood of achieving the negative status for MRD is about 24% or higher.

MRD status may be assessed from bone marrow aspirate samples using for example next generation sequencing (NGS) of immunoglobulin heavy and light chains. The updated, analytically validated version of the clonoSEQ® Assay (Version 2) by Adaptive Biotechnologies may be used for the detection, quantification and analysis of MRD.

The disclosure also provides a method of treating a subject with newly diagnosed multiple myeloma, comprising administering to the subject a combination therapy demonstrated to increase a likelihood of achieving a CR or better in subjects with newly diagnosed multiple myeloma, wherein the combination therapy comprises daratumumab, lenalidomide and dexamethasone.

In some embodiments, the likelihood of achieving the CR or better is about 47% or higher.

The disclosure also provides a method of treating a subject with newly diagnosed multiple myeloma, comprising administering to the subject a combination therapy demonstrated to reduce a risk of progression of multiple myeloma or death in subjects with newly diagnosed multiple myeloma, wherein the combination therapy comprises daratumumab, lenalidomide and dexamethasone.

In some embodiments, the risk of progression of multiple myeloma or death is reduced by about 44%.

In some embodiments, the subject with newly diagnosed multiple myeloma is ineligible for autologous stem cell transplant (ASCT).

In eligible subjects, ASCT is provided in conjunction with high dose chemotherapy (HDC) as described herein.

In some embodiments, the combination therapy comprises about 16 mg/kg daratumumab, about 25 mg lenalidomide and between about 20 mg and about 40 mg dexamethasone.

In some embodiments, the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

In some embodiments, dexamethasone is administered as pre-medication on daratumumab administration days.

Dexamethasone may be administered about 20 mg the day of daratumumab administration and 20 mg a day after daratumumab administration.

In some embodiments, daratumumab is administered intravenously, lenalidomide is administered orally and dexamethasone is administered intravenously or orally.

In some embodiments, lenalidomide, dexamethasone or both lenalidomide and dexamethasone are self-administered.

In some embodiments, daratumumab is provided for administration by a manufacturer of daratumumab in a single-dose vial comprising 100 mg daratumumab in 5 mL of solution or in a single-dose vial comprising 400 mg daratumumab in 20 mL of solution.

In some embodiments, each single-dose vial comprising 100 mg daratumumab in 5 mL of solution and each single-dose vial comprising 400 mg daratumumab in 20 mL of solution further comprises glacial acetic acid, mannitol, polysorbate 20, sodium acetate trihydrate and sodium chloride.

In some embodiments, each single-dose vial comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and each single-dose vial comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

In some embodiments, daratumumab is diluted into 0.9% sodium chloride prior to administration.

In some embodiments, information that a combination therapy comprising daratumumab, lenalidomide and dexamethasone is safe and effective is provided on a daratumumab-containing drug product label or package insert.

Exemplary information is clinical trial results from an open-label, randomized active-controlled phase 3 study MAIA, listed at ClinicalTrials_gov database as study NCT02252172.

In some embodiments, the daratumumab-containing drug product label includes information that a recommended dose of daratumumab is 16 mg/kg administered as an intravenous injection.

In some embodiments, the daratumumab-containing drug product label includes information that the recommended dosing schedule of daratumumab in combination with lenalidomide is once a week on weeks 1 to 8, once in two weeks on weeks 9-24 and thereafter once in four weeks.

In some embodiments, the daratumumab-containing drug product label includes information that the recommended dosing schedule of lenalidomide is 25 mg daily on days 1-21 of repeated 4 week cycles.

In some embodiments, the daratumumab-containing drug product label includes information that the recommended dosing schedule of dexamethasone is about 20 mg or about 40 mg per week.

In some embodiments, daratumumab, lenalidomide and dexamethasone are administered according to the recommended dosing schedules.

In some embodiments, the daratumumab-containing drug product label includes data from an open-label, randomized active-controlled phase 3 study that compared treatment with daratumumab, lenalidomide and dexamethasone (DRd) to treatment with lenalidomide and dexamethasone (Rd) in subjects with newly diagnosed multiple myeloma who are ineligible for ASCT.

In some embodiments, the open-label, randomized active-controlled phase 3 study is known as MAIA, listed at ClinicalTrials_gov database as study NCT02252172.

In some embodiments, the daratumumab-containing drug product label includes data that treatment with DRd resulted in about 44% reduction in the risk of multiple myeloma progression or death when compared to treatment with Rd.

In some embodiments, the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

In some embodiments, the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

In some embodiments, the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

In some embodiments, the phase 3 active-controlled study is known as ALCYONE, listed at ClinicalTrials_gov database as study NCT02195479.

In some embodiments, the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the daratumumab-containing drug product label includes data from a phase 3 active-controlled study that compared treatment with daratumumab, bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the daratumumab-containing drug product label includes drug interaction data informing that clinical pharmacokinetic assessments of daratumumab in combination with lenalidomide, pomalidomide bortezomib and dexamethasone indicated no clinically relevant drug-drug interactions between daratumumab and lenalidomide, pomalidomide, bortezomib and dexamethasone.

In some embodiments, the daratumumab-containing drug product label includes information that side effects of daratumumab include weakness, decreased appetite, bronchitis and lung infection.

In some embodiments, the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a heavy chain variable region (VH) of SEQ ID NO: 7 and a light chain variable region (VL) of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as G1m17, G1m3, G1m1, G1m2, G1m27 or G1m28.

In some embodiments, daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line. In some embodiments, the mammalian cell line is a Hek cell line.

In some embodiments, the molecular weight of daratumumab is about 148 kDa.

In some embodiments, dexamethasone can be substituted for a dexamethasone equivalent, wherein the dexamethasone equivalent is methylprednisolone, prednisolone, prednisone or betamethasone, or any combination thereof.

The disclosure also provides a method of treating a subject with newly diagnosed multiple myeloma, comprising:

providing a healthcare professional (HCP) daratumumab;
providing the HCP information that treating the subject with a combination therapy comprising daratumumab, lenalidomide and dexamethasone achieves an improved clinical efficacy endpoint when compared to a clinical efficacy endpoint achieved if the subject were treated with a combination of lenalidomide and dexamethasone; wherein performing the steps a) and b) results in the subject with newly diagnosed multiple myeloma to receive the combination therapy comprising daratumumab, lenalidomide and dexamethasone by the HCP or by self-administration as instructed by the HCP, thereby treating the subject having the newly diagnosed multiple myeloma.

The disclosure also provides a method of providing daratumumab to a HCP for the HCP to treat a subject with newly diagnosed multiple myeloma with a combination therapy comprising daratumumab, lenalidomide and dexamethasone, wherein the treatment with the combination therapy comprising daratumumab, lenalidomide and dexamethasone achieves an improved clinical efficacy endpoint when compared to a clinical efficacy endpoint achieved if the subject were treated with a combination of lenalidomide and dexamethasone, comprising:

manufacturing daratumumab;
providing the HCP information that treatment with the combination therapy comprising daratumumab, lenalidomide and dexamethasone achieves the improved clinical efficacy endpoint; and
shipping daratumumab to the HCP or to an authorized distributor of daratumumab for the HCP to purchase daratumumab; thereby providing daratumumab to the HCP.

The disclosure also provides a method of providing a treatment option for a HCP to treat a subject with newly diagnosed multiple myeloma with a combination therapy comprising daratumumab, lenalidomide and dexamethasone, wherein the treatment with the combination therapy comprising daratumumab, lenalidomide and dexamethasone achieves an improved clinical efficacy endpoint when compared to a clinical efficacy endpoint achieved if the subject were treated with a combination of lenalidomide and dexamethasone, comprising:

manufacturing daratumumab;
providing the HCP information that the combination therapy comprising daratumumab, lenalidomide and dexamethasone achieves the improved clinical efficacy endpoint; and
shipping daratumumab to the HCP or to an authorized distributor of daratumumab for the HCP to purchase daratumumab, thereby providing the treatment option for the HCP.

Exemplary information is clinical trial results from an open-label, randomized active-controlled phase 3 study known as MAIA, listed at ClinicalTrials_gov database as a study NCT02252172.

In some embodiments, the subject is ineligible for autologous stem cell transplant (ASCT).

In eligible subjects, ASCT is provided in conjunction with high dose chemotherapy (HDC) as described herein.

In some embodiments, the combination therapy comprising daratumumab, lenalidomide and dexamethasone is demonstrated to increase a likelihood of achieving a VGPR or better in subjects with newly diagnosed multiple myeloma.

In some embodiments, the likelihood of achieving the VGPR or better is about 79% or higher.

In some embodiments, the combination therapy comprising daratumumab, lenalidomide and dexamethasone is demonstrated to increase a likelihood of achieving a negative status for MRD in subjects with newly diagnosed multiple myeloma.

In some embodiments, the likelihood of achieving the negative status for MRD is about 24% or higher.

In some embodiments, the combination therapy comprising daratumumab, lenalidomide and dexamethasone is demonstrated to increase a likelihood of achieving a CR or better in subjects with newly diagnosed multiple myeloma.

In some embodiments, the likelihood of achieving the CR or better is about 47% or higher.

In some embodiments, the combination therapy comprising daratumumab, lenalidomide and dexamethasone is demonstrated to reduce a risk of progression of multiple myeloma or death in subjects with newly diagnosed multiple myeloma.

In some embodiments, the risk of progression of multiple myeloma or death is reduced by about 44%.

In some embodiments, the combination therapy comprises about 16 mg/kg daratumumab, about 25 mg lenalidomide and between about 20 mg and about 40 mg dexamethasone.

In some embodiments, the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4 week cycles, and about 20 mg to about 40 mg dexamethasone administered per week.

In some embodiments, the combination therapy comprises administering dexamethasone as pre-medication on daratumumab administration days.

Dexamethasone may be administered about 20 mg the day of daratumumab administration and 20 mg a day after daratumumab administration.

In some embodiments, the combination therapy comprises administering daratumumab intravenously, lenalidomide orally and dexamethasone intravenously or orally.

In some embodiments, daratumumab is shipped or provided by a manufacturer of daratumumab in a single-dose vial comprising 100 mg daratumumab in 5 mL of solution or in a single-dose vial comprising 400 mg daratumumab in 20 mL of solution.

In some embodiments, each single-dose vial comprising 100 mg daratumumab in 5 mL of solution and each single-dose vial comprising 400 mg daratumumab in 20 mL of solution further comprises glacial acetic acid, mannitol, polysorbate 20, sodium acetate trihydrate and sodium chloride.

In some embodiments, each single-dose vial comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and each single-dose vial comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

In some embodiments, daratumumab is diluted into 0.9% sodium chloride prior to administration.

In some embodiments, information that the combination therapy comprising daratumumab, lenalidomide and dexamethasone achieves the improved clinical efficacy endpoint is provided on a daratumumab-containing drug product label.

In some embodiments, the daratumumab-containing drug product label includes information that a recommended dose of daratumumab is 16 mg/kg administered as an intravenous infusion.

In some embodiments, the daratumumab-containing drug product label includes information that the recommended dosing schedule of daratumumab in combination with lenalidomide is once a week on weeks 1 to 8, once in two weeks on weeks 9-24 and thereafter once in four weeks.

In some embodiments, the daratumumab-containing drug product label includes information that the recommended dosing schedule of lenalidomide is 25 mg once daily on days 1-21 of repeated 4-week cycles.

In some embodiments, the daratumumab-containing drug product label includes information that the recommended dosing schedule of dexamethasone is about 20 mg or about 40 mg per week.

In some embodiments, daratumumab, lenalidomide and dexamethasone are administered according to the recommended dosing schedules.

In some embodiments, the daratumumab-containing drug product label includes data from an open-label, randomized active-controlled phase 3 study that compared treatment with daratumumab, lenalidomide and dexamethasone (DRd) to treatment with lenalidomide and dexamethasone (Rd) in subjects with newly diagnosed multiple myeloma who are ineligible for ASCT.

In some embodiments, the open-label, randomized active-controlled phase 3 study is known as MAIA, listed at ClinicalTrials_gov database as study NCT02252172.

In some embodiments, the daratumumab-containing drug product label includes data that treatment with DRd resulted in about 44% reduction in the risk of multiple myeloma progression or death when compared to treatment with Rd.

In some embodiments, the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

In some embodiments, the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

In some embodiments, the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

In some embodiments, the phase 3 active-controlled study is known as ALCYONE, listed at ClinicalTrials_gov database as a study NCT02195479.

In some embodiments, the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the daratumumab-containing drug product label includes data from a phase 3 active-controlled study that compared treatment with daratumumab, bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the daratumumab-containing drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

In some embodiments, the daratumumab-containing drug product label includes information that side effects of daratumumab includes feeling weak, decreased appetite, bronchitis and lung infection.

In some embodiments, the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6.

In some embodiments, daratumumab comprises a VH of SEQ ID NO: 7 and a VL of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as G1m17, G1m3, G1m1, G1m2, G1m27 or G1m28.

In some embodiments, daratumumab comprises a HC of SEQ ID NO: 9 and a LC of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

In some embodiments, dexamethasone can be substituted for dexamethasone equivalent, wherein dexamethasone equivalent is methylprednisolone, prednisolone, prednisone or betamethasone, or any combination thereof.

### Combination Therapies and Drug Products of the Disclosure

The disclosure also provides a combination therapy comprising daratumumab, lenalidomide and dexamethasone for providing a treatment of a subject with newly diagnosed multiple myeloma, wherein the treatment achieves an improved clinical efficacy endpoint when compared to a clinical efficacy endpoint achieved if the subject were treated with a combination of lenalidomide and dexamethasone.

In some embodiments, the combination therapy of the disclosure comprises about 16 mg/kg daratumumab, about 25 mg lenalidomide and about 20 mg to about 40 mg dexamethasone.

In some embodiments, the treatment of the subject with newly diagnosed multiple myeloma comprises administering to the subject about 16 mg/kg daratumumab once a week, once in two weeks or once in four weeks, about 25 mg lenalidomide daily and about 20 mg to about 40 mg dexamethasone per week.

In some embodiments, the treatment of the subject with newly diagnosed multiple myeloma comprises administering to the subject about 16 mg/kg daratumumab once a week on weeks 1-8, once in two weeks on weeks 9-24 and once in four weeks thereafter, about 25 mg lenalidomide once daily on days 1-21 of repeated 4-week cycles and about 20 mg or about 40 mg per week dexamethasone.

In some embodiments, the combination therapy is demonstrated to increase a likelihood of achieving a VGPR or better in subjects with newly diagnosed multiple myeloma.

In some embodiments, the likelihood of achieving the VGPR or better is about 79% or more.

In some embodiments, the combination therapy is demonstrated to increase a likelihood of achieving a negative status for MRD in subjects with newly diagnosed multiple myeloma.

In some embodiments, the likelihood of achieving the negative status for MRD is about 24% or more.

In some embodiments, the combination therapy is demonstrated to increase a likelihood of achieving a CR or better in subjects with newly diagnosed multiple myeloma.

In some embodiments, the likelihood of achieving the CR or better is about 47% or more.

In some embodiments, the combination therapy is demonstrated to reduce a risk of progression of multiple myeloma or death in subjects with newly diagnosed multiple myeloma.

In some embodiments, the risk of progression of multiple myeloma or death is reduced by about 44%.

In some embodiments, the subject with multiple myeloma is ineligible for autologous stem cell transplant (ASCT).

In some embodiments, the combination therapy is promoted by a manufacturer of daratumumab for treatment of newly diagnosed multiple myeloma. Promotion may be in a form of any published record demonstrating that the treatment is safe and effective and approved by the FDA, such as product claim advertisements, either in print or broadcast, promotional labeling including brochures and materials mailed or provided to consumers, and other types of materials given out by manufacturer of a daratumumab-containing drug product, including drug product label and prescribing information.

In some embodiments, the combination therapy is promoted by a manufacturer of the daratumumab-containing drug product for treatment of newly diagnosed multiple myeloma on a daratumumab-containing drug product label.

In some embodiments, the daratumumab-containing drug product label includes data from an open-label, randomized active-controlled phase 3 study that compared treatment with daratumumab in combination with lenalidomide and dexamethasone (DRd) to treatment with lenalidomide and dexamethasone (Rd) in patients with newly diagnosed multiple myeloma.

In some embodiments, the open-label, randomized active-controlled phase 3 study is known as MAIA, listed at ClinicalTrials_gov database as study NCT02252172.

In some embodiments, the daratumumab-containing drug product label includes data that treatment with DRd resulted in about 44% reduction in the risk of multiple myeloma progression or death when compared to treatment with Rd.

In some embodiments, the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

In some embodiments, the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

In some embodiments, the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

In some embodiments, the phase 3 active-controlled study is known as ALCYONE, listed at ClinicalTrials_gov database as study NCT02195479.

In some embodiments, the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the daratumumab-containing drug product label includes data from a phase 3 active-controlled study that compared treatment with daratumumab, bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the daratumumab-containing drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

In some embodiments, the daratumumab-containing drug product label includes information that side effects of daratumumab includes weakness, decreased appetite, bronchitis and lung infection.

In some embodiments, the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6.

In some embodiments, daratumumab comprises a VH of SEQ ID NO: 7 and a VL of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles;

allotypes). The antibody that specifically binds CD38 may be of any IgG1 allotype, such as G1m17, G1m3, G1m1, G1m2, G1m27 or G1m28.

In some embodiments, daratumumab comprises a HC of SEQ ID NO: 9 and a LC of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

In some embodiments, dexamethasone can be substituted for a dexamethasone equivalent, wherein the dexamethasone equivalent is methylprednisolone, prednisolone, prednisone or betamethasone, or any combination thereof.

The disclosure also provides a drug product comprising daratumumab that is provided in a package comprising one or more single-dose vials comprising daratumumab and a drug product label that includes information that treatment of a subject with newly diagnosed multiple myeloma with a combination therapy comprising daratumumab, lenalidomide and dexamethasone achieves an improved clinical efficacy endpoint when compared to a clinical efficacy endpoint achieved if the subject were administered a combination of lenalidomide and dexamethasone.

In some embodiments, the one or more single-dose vials comprises 100 mg daratumumab in 5 mL of solution or 400 mg daratumumab in 20 mL of solution.

In some embodiments, the one or more single-dose vials comprising 100 mg daratumumab in 5 mL of solution and the one or more single-dose vials comprising 400 mg daratumumab in 20 mL of solution further comprises glacial acetic acid, mannitol, polysorbate 20, sodium acetate trihydrate and sodium chloride.

In some embodiments, the one or more single-dose vials comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and the one or more single-dose vials comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

In some embodiments, the drug product label includes information that a recommended dosing schedule of daratumumab is 16 mg/kg once a week on weeks 1 to 8, once in two weeks on weeks 9-24 and thereafter once in four weeks, the recommended dosing schedule of lenalidomide is 25 mg daily on days 1-21 of repeated 4 week cycles, and the recommended dosing schedule of dexamethasone is 20 mg per week or 40 mg per week.

In some embodiments, the drug product label includes data from an open-label, randomized active-controlled phase 3 study that compared treatment with daratumumab in combination with lenalidomide and dexamethasone (DRd) to treatment with lenalidomide and dexamethasone (Rd) in patients with newly diagnosed multiple myeloma.

In some embodiments, the open-label, randomized active-controlled phase 3 study is known as MAIA, listed at ClinicalTrials_gov database as study NCT02252172.

In some embodiments, the drug product label includes data that treatment with DRd resulted in about 44% reduction in the risk of multiple myeloma progression or death when compared to treatment with Rd.

In some embodiments, the drug product label includes data that treatment with DRd resulted in about 79.3% of subjects achieving VGPR or better, about 24% of subjects achieving a negative status for MRD, or about 47.6% of subjects achieving CR or better, or any combination thereof.

In some embodiments, the drug product label includes a Kaplan-Meier curve of progression-free survival (PFS) comparing subjects having newly diagnosed multiple myeloma treated with DRd to subjects having newly diagnosed multiple myeloma treated with Rd.

In some embodiments, the drug product label includes data from a phase 3 active-controlled study that compared treatment with daratumumab, bortezomib, melphalan and prednisone (D-VMP) to treatment with bortezomib, melphalan and prednisone (VMP).

In some embodiments, the phase 3 active-controlled study is known as ALCYONE, listed at ClinicalTrials_gov database as study NCT02195479.

In some embodiments, the drug product label includes data from a phase 3 active-controlled study that compared treatment with daratumumab in combination with lenalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the drug product label includes data from a phase 3 active-controlled study that compared treatment with daratumumab in combination with bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the drug product label includes drug product interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

In some embodiments, the drug product label includes information that side effects of daratumumab includes weakness, decreased appetite, bronchitis and lung infection.

In some embodiments, the drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6.

In some embodiments, daratumumab comprises a VH of SEQ ID NO: 7 and a VL of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as G1m17, G1m3, G1m1, G1m2, G1m27 or G1m28.

In some embodiments, daratumumab comprises a HC of SEQ ID NO: 9 and a LC of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

The disclosure also provides a method of selling a drug product comprising daratumumab, comprising:

manufacturing daratumumab;
promoting that a combination therapy comprising daratumumab, lenalidomide and dexamethasone achieves an improved clinical efficacy endpoint when administered to a subject with newly diagnosed multiple myeloma, when compared to a clinical efficacy endpoint achieved if the subject were administered a combination of lenalidomide and dexamethasone, wherein performing the steps a) and b) results in a HCP to purchase the drug product; thereby selling the drug product.

Promotion may be in a form of any published record demonstrating that the treatment is safe and effective and approved by the FDA, such as product claim advertisements, either in print or broadcast, promotional labeling including brochures and materials mailed or provided to consumers, and other types of materials given out by manufacturer of daratumumab, including drug product label and prescribing information.

In some embodiments, promoting comprises including data from an open-label, randomized active-controlled phase 3 study that compared treatment with daratumumab in combination with lenalidomide and dexamethasone (DRd) to treatment with lenalidomide and dexamethasone (Rd) in patients with newly diagnosed multiple myeloma on the drug product label.

In some embodiments, the drug product label further includes data that treatment with DRd resulted in about 44% reduction in the risk of multiple myeloma progression or death when compared to treatment with Rd.

In some embodiments, the drug product label further includes a Kaplan-Meier curve of progression-free survival (PFS) comparing subjects having newly diagnosed multiple myeloma treated with DRd to subjects having newly diagnosed multiple myeloma treated with Rd.

The invention also provides a method of selling a drug product comprising daratumumab, comprising

manufacturing daratumumab;
selling the drug product, wherein the drug product label includes an indication for treating a subject with newly diagnosed multiple myeloma with a combination of daratumumab, lenalidomide and dexamethasone.

In some embodiments, daratumumab is DARZALEX® brand of daratumumab.

In some embodiments, daratumumab is a biosimilar of DARZALEX® brand of daratumumab.

In some embodiments, daratumumab comprises a HCDR1 of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a VH of SEQ ID NO: 7 and a VL of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as G1m17, G1m3, G1m1, G1m2, G1m27 or G1m28.

In some embodiments, daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

### Methods of Producing Antibodies

Methods of producing antibodies at large scales are known. Antibodies may be produced for example in CHO cells cultured using known methods. The antibody may be isolated and/or purified from culture medium by removing solids by centrifugation or filtering as a first step in the purification process. The antibody may be further purified by standard methods including chromatography (e.g., ion exchange, affinity, size exclusion, and hydroxyapatite chromatography), gel filtration, centrifugation, or differential solubility, ethanol precipitation or by any other available technique for the purification of antibodies. Protease inhibitors such as phenyl methyl sulfonyl fluoride (PMSF), leupeptin, pepstatin or aprotinin can be added at any or all stages in order to reduce or eliminate degradation of the antibody during the purification process. One of ordinary skill in the art will appreciate that the exact purification technique will vary depending on the character of the polypeptide or protein to be purified, the character of the cells from which the polypeptide or protein is expressed, and the composition of the medium in which the cells were grown.

The purified antibody is formulated in a pharmaceutical composition comprising one or more excipients and packaged into a container such as a sealed bottle or vessel, such as a glass vial, with label affixed to the container or included in the package. Alternatively, the purified antibody may be lyophilized and provided as a lyophilized powder in the container.

While having described the invention in general terms, the embodiments of the invention will be further disclosed in the following examples that should not be construed as limiting the scope of the claims.

### Example 1: Phase 3 Study Comparing DARZALEX® (Daratumumab), Lenalidomide and Dexamethasone (DRd) Vs. Lenalidomide and Dexamethasone (Rd) in Subjects with Previously Untreated Multiple Myeloma Who are Ineligible for High Dose Chemotherapy (HDC) and Autologous Stem Cell Transplant (ASCT)

### Objectives and Hypothesis

### Primary Objective

The primary objective is to compare the efficacy of daratumumab (DARZALEX®) when combined with lenalidomide and dexamethasone (DRd) to that of lenalidomide and dexamethasone (Rd), in terms of progression-free survival (PFS) in subjects with newly diagnosed myeloma who are not candidates for high dose chemotherapy (HDC) and autologous stem cell transplant (ASCT).

### Secondary Objectives

The secondary objectives are:
To evaluate clinical outcomes including:
   Time to disease progression (TTP)
   CR rate
   MRD negativity rate
   PFS2 (defined as time from randomization to progression on the next line of therapy or death, whichever comes first)
   Overall survival
   Time to next treatment
   Stringent CR (sCR) rate
   Overall response rate (partial response [PR] or better)
   Proportion of subjects who achieve very good partial response (VGPR) or better
   Time to response
   Duration of response
To assess the safety and tolerability of daratumumab (DARZALEX®) when administered in combination with Rd
To assess the pharmacokinetics of daratumumab (DARZALEX®) in combination with Rd
To assess the immunogenicity of daratumumab (DARZALEX®)
To evaluate treatment effects on patient reported outcomes and heath economic/resource utilization
To evaluate the clinical efficacy of daratumumab (DARZALEX®) combination with Rd in high-risk molecular subgroups

### Exploratory Objectives

To explore biomarkers predictive of response or resistance to therapy
To assess durability of MRD negativity

### Overview of Study Design

This is a randomized, open-label, active controlled, parallel-group, multicenter study in subjects at least 18 years of age with newly diagnosed multiple myeloma who are not candidates for HDC and ASCT. Approximately 730 subjects will be enrolled in this study with 365 subjects planned per treatment arm.

Subject participation will include a Screening Phase, a Treatment Phase, and a Follow-up Phase. The Screening Phase will be up to 21 days before Cycle 1, Day 1. The Treatment Phase will extend from Day 1 of Cycle 1 until discontinuation of all study treatment. For subjects assigned to DRd, daratumumab (DARZALEX®) will be administered weekly for the first 8 weeks (Cycles 1-2) of treatment and then every other week for 16 weeks (Cycles 3-6), then every 4 weeks (from Cycle 7 and beyond) until disease progression or unacceptable toxicity. This will equate to 9 consecutive weeks of dosing at the start of the study and a total of 23 doses in the first year. Lenalidomide will be administered at a dose of 25 mg orally (PO) on Days 1 through 21 of each 28-day cycle, and dexamethasone will be administered at a dose of 40 mg once a week. Subjects in both treatment arms will continue lenalidomide and dexamethasone until disease progression or unacceptable toxicity. Subjects in the DRd arm will continue on daratumumab)(DARZALEX® until disease progression or unacceptable toxicity. Randomization will be stratified by International Staging System (I vs II vs III), region (North America vs Other), and age (<75 vs >75), using an equal allocation ratio of 1:1.

Measures to prevent infusion-related reactions will include preinfusion medication with dexamethasone, acetaminophen (paracetamol), and an antihistamine before each daratumumab (DARZALEX®) infusion.

The Follow-up Phase will begin once a subject discontinues all study treatments. Subjects who discontinue for reasons other than disease progression must continue to have disease evaluations according to the Time and Events Schedule. The Follow-up Phase will continue until death, lost to follow up, consent withdrawal, or study end, whichever occurs first. After the clinical cut-off, data collection will be reduced.

An Independent Data Monitoring Committee (IDMC) will be commissioned for this study to review efficacy and safety results at planned interim analyses. After the interim review, the IDMC will make recommendations regarding the continuation of the study.

Assessment of tumor response and disease progression will be conducted in accordance with the International Myeloma Working Group (IMWG) response criteria. An assessment of MRD will be conducted on bone marrow samples. Safety evaluations will include adverse event monitoring, physical examinations, electrocardiogram (ECG) monitoring, clinical laboratory parameters (hematology and chemistry), vital sign measurements, and Eastern Cooperative Oncology Group (ECOG) performance status. Blood samples will be drawn for assessment of pharmacokinetic parameters.

### Subject Population

Key eligibility criteria include the following: subjects who are 18 years of age, have a confirmed diagnosis of symptomatic multiple myeloma and measurable secretory disease, an ECOG performance status score of 0, 1, or 2, must be newly diagnosed and not considered candidates for high-dose chemotherapy (HDC) with autologous stem cell transplantation (ASCT).

### Dosage and Administration

Daratumumab (DARZALEX®) (16 mg/kg) will be administered by IV infusion to subjects in Arm B initially once every week for 8 weeks; then once every other week for 16 weeks; thereafter once every 4 weeks until documented progression, unacceptable toxicity, or study end.

Lenalidomide will be self-administered at a dose of 25 mg PO each day on Days 1 through 21 of each 28-day cycle.

Dexamethasone (or equivalent in accordance with local standards) will be administered at a total dose of 40 mg weekly.

### Efficacy Evaluations/Endpoints

Disease evaluations must be performed every 28 days for the first 2 years and then every 8 weeks until disease progression. A window of ±7 days is allowed. If treatment has been delayed for any reason, the disease evaluations must be performed according to schedule, regardless of any changes to the dosing regimen.

The primary endpoint is PFS, which is defined as the duration from the date of randomization to either progressive disease, or death, whichever occurs first. Disease progression will be determined according to the IMWG criteria.

The secondary efficacy endpoints include:
Time to disease progression (TTP) is defined as the time from the date of randomization to the date of first documented evidence of PD, as defined in the IMWG criteria. For subjects who have not progressed, data will be censored at the date of the disease evaluation before the start of any subsequent anti-myeloma therapy.
CR rate, defined as the percentage of subjects achieving CR, as defined:
   Negative immunofixation of serum and urine, and
   Disappearance of any soft tissue plasmacytomas, and
   <5% plasma cells (PCs) in bone marrow
   For those subjects with negative serum M-protein quantitation by electrophoresis (SPEP) and suspected daratumumab (DARZALEX®) interference on immunofixation, a reflex assay using anti-idiotype antibody will be utilized to confirm daratumumab (DARZALEX®) interference and rule out false positive immunofixation. Patients who have confirmed daratumumab (DARZALEX®) interference, but meet all other clinical criteria for CR or sCR, will be considered CR/sCR.
MRD negativity rate, defined as the proportion of subjects assessed as MRD negative, at any timepoint after the date of randomization.
Progression-free Survival on Next line of Therapy (PFS2), defined as the time from randomization to progression on the next line of treatment or death, whichever comes first. Disease progression will be based on investigator judgment. For those subjects who are still alive and not yet progressed on the next line of treatment, they will be censored on the last date of follow-up.
Overall survival (OS), measured from the date of randomization to the date of the subject's death. If the subject is alive or the vital status is unknown, then the subject's data will be censored at the date the subject was last known to be alive.
Time to next treatment, defined as the time from randomization to the start of the next-line treatment.
sCR rate, defined as the percentage of subjects achieving CR in addition to having a normal free light chain (FLC) ratio and an absence of clonal cells in bone marrow by immunohistochemistry, immunofluorescence, 2-4 color flow cytometry.
Overall response rate (ORR), defined as the proportion of subjects who achieve PR or better, according to the IMWG criteria, during or after the study treatment.
Proportion of subjects who achieve VGPR or better, defined as the proportion of subjects achieving VGPR and CR (including sCR) according to the IMWG criteria during or after the study treatment at the time of data cut-off
Time to response, defined as the time between the randomization and the first efficacy evaluation that the subject has met all criteria for PR or better. For subjects without response, data will be censored either at the date of progressive disease or, in the absence of progressive disease, at the last disease evaluation before the start of subsequent anti-myeloma therapy.
Duration of response, calculated from the date of initial documentation of a response (PR or better) to the date of first documented evidence of progressive disease, as defined in the IMWG criteria. For subjects who have not progressed, data will be censored at the last disease evaluation before the start of any subsequent anti-myeloma therapy.
To evaluate clinical efficacy of DRd in high risk molecular subgroups compared to Rd alone.
To evaluate the impact of DRd compared to Rd on patient-reported perception of global health.

### Pharmacokinetic and Immunogenicity Evaluations

For all subjects in Arm B, pharmacokinetic samples to determine serum concentration of daratumumab (DARZALEX®) will be obtained. Venous blood samples (5 mL per sample) will be collected to determine serum concentration of daratumumab (DARZALEX®) and the serum will be divided into 3 aliquots (1 aliquot for pharmacokinetic analysis, 1 aliquot for antibodies to daratumumab (DARZALEX®) analysis when appropriate, and 1 aliquot as a backup).

### Biomarker Evaluations

Bone marrow aspirates will be collected at screening and following treatment. Baseline bone marrow aspirate samples will be subjected to DNA and RNA sequencing in order to classify subjects into high-risk molecular subgroups and to establish the myeloma clone for MRD monitoring. In addition to planned bone marrow aspirate assessments, a whole blood sample will be collected from subjects for processing to plasma and PBMCs.

### Safety Evaluations

Safety will be measured by adverse events, laboratory test results, ECGs, vital sign measurements, physical examination findings, and assessment of ECOG performance status score.

### Statistical Methods

The sample size calculation is performed on the basis of the following assumption. Based on the published data, the median PFS for Rd arm is assumed to be approximately 24 months. Assuming that DRd can reduce the risk of the disease progression or death by 25%, i.e., assuming the hazard ratio (DRd vs Rd) of 0.75, a total of 390 PFS events is needed to achieve a power of 80% to detect this hazard ratio with a log-rank test (two-sided alpha is 0.05). With a 21-month accrual period and an additional 24-month follow-up, the total sample size needed for the study is approximately 730 (365/arm) subjects. The sample size calculation has taken into consideration an annual dropout rate of 5%.

Long-term survival follow-up will continue until 330 deaths have been observed or 7 years after the last subject is randomized. Therefore, this study will achieve approximately 80% power to detect a 27% reduction in the risk of death (hazard ratio=0.73) with a log-rank test (two-sided alpha=0.05).

Response to study treatment and progressive disease will be evaluated by a computer algorithm. For the primary endpoint of PFS, the primary analysis will consist of a stratified log rank test for the comparison of the PFS distribution between the 2 treatment arms. The Kaplan-Meier method will be used to estimate the distribution of overall PFS for each treatment. The treatment effect (hazard ratio) and its two-sided 95% confidence intervals are to be estimated using a stratified Cox regression model with treatment as the sole explanatory variable.

### Rationale for DNA and Biomarker Collection

Biomarker samples will be collected to evaluate the depth of clinical response to daratumumab (DARZALEX®) through evaluation of MRD, using DNA sequencing of immunoglobulin genes, and to determine response rates in specific molecular subgroups of multiple myeloma, using DNA/RNA sequencing of multiple myeloma cells to allow for assessment of high-risk genomics such as deletion 17p, t(4;14), t(14;20), t(14;16), deletion13, GEP signatures such as UAMS-70, and mutations in p53, BRAF, FGFR, IGH, PI3K, or other molecular subtypes associated with disease progression. Other biomarker goals include evaluation of potential mechanisms of resistance, inter-individual variability in clinical outcomes or identification of population subgroups that respond differently to treatment.

### Inclusion Criteria

Each potential subject must satisfy all of the following criteria to be enrolled in the study.
1. Subject must be at least 18 years of age (or the legal age of consent in the jurisdiction in which the study is taking place).
2. 2.1 Subject must have documented multiple myeloma satisfying the CRAB (calcium elevation, renal insufficiency, anemia and bone abnormalities) criteria, monoclonal plasma cells in the bone marrow ≥10% or presence of a biopsy proven plasmacytoma, and measurable disease.
   Measurable disease, as assessed by central laboratory, defined by any of the following:
      IgG myeloma: Serum monoclonal paraprotein (M-protein) level ≥1.0 g/dL or urine M-protein level ≥200 mg/24 hours; or
      IgA, IgM, IgD, or IgE multiple myeloma: serum M-protein level ≥0.5 g/dL or urine M-protein level ≥200 mg/24 hours; or
      Light chain multiple myeloma without measurable disease in serum or urine: Serum immunoglobulin free light chain ≥10 mg/dL and abnormal serum immunoglobulin kappa lambda free light chain ratio.
3. Newly diagnosed and not considered candidate for high-dose chemotherapy with SCT due to:
   Being age ≥65 years, OR
   In subjects ≤65 years: presence of important comorbid condition(s) likely to have a negative impact on tolerability of high dose chemotherapy with stem cell transplantation. Sponsor review and approval of subjects under 65 years of age is required before randomization.
4. Subject must have an ECOG performance status score of 0, 1, or 2
5. Subject must have pretreatment clinical laboratory values meeting the following criteria during the Screening Phase:
   a) hemoglobin 7.5 g/dL (≥5 mM/L; prior red blood cell [RBC] transfusion or recombinant human erythropoietin use is permitted);
   b) absolute neutrophil count ≥1.0×10⁹/L (granulocyte colony stimulating factor [GCSF] use is permitted);
   c) platelet count 70×10⁹/L for subjects in whom ≤50% of bone marrow nucleated cells are plasma cells; otherwise platelet count ≥50×10⁹/L (transfusions are not permitted to achieve this minimum platelet count);
   d) aspartate aminotransferase (AST)≤2.5× upper limit of normal (ULN);
   e) alanine aminotransferase (ALT)≤2.5×ULN;
   f) total bilirubin ≤2.0×ULN, except in subjects with congenital bilirubinemia, such as Gilbert syndrome (direct bilirubin ≤2.0×ULN);
      g1) Creatinine clearance ≥30 mL/min (for lenalidomide dose adjustment for subjects with creatinine clearance 30-50 mL/min). Creatinine clearance can be calculated using the Cockcroft-Gault formula; or for subjects with over- or underweight, creatinine clearance may be measured from a 24-hours urine collection
      h1) corrected serum calcium ≤14 mg/dL (≤3.5 mM/L); or free ionized calcium ≤6.5 mg/dL (≤1.6 mM/L)
6. Contraceptive use by men or women should be consistent with local regulations regarding the use of contraceptive methods for subjects participating in clinical studies. Women of childbearing potential must commit to either abstain continuously from heterosexual sexual intercourse or to use 2 methods of reliable birth control simultaneously. This includes one highly effective form of contraception (tubal ligation, intrauterine device (IUD), hormonal (progesterone-only birth control pills or injections) or partner's vasectomy and one additional effective contraceptive method (male latex or synthetic condom, diaphragm, or cervical cap). Contraception must begin 4 weeks prior to dosing and must continue for 3 months after the last dose of daratumumab) (DARZALEX®. Reliable contraception is indicated even where there has been a history of infertility, unless due to hysterectomy or bilateral oophorectomy.
7. A man who is sexually active with a woman of childbearing potential must agree to use a latex or synthetic condom, even if he had a successful vasectomy. All men must also not donate sperm during the study, for 4 weeks after the last dose of lenalidomide, and for 3 months after the last dose of daratumumab (DARZALEX®).
8. A woman of childbearing potential must have 2 negative serum or urine pregnancy tests at screening, first within 10 to 14 days prior to dosing and the second within 24 hours prior to dosing.
9. Each subject (or their legally acceptable representative) must sign an informed consent form (ICF) indicating that he or she understands the purpose of and procedures required for the study and are willing to participate in the study. Subject must be willing and able to adhere to the prohibitions and restrictions specified in this protocol, as referenced in the ICF.

### Exclusion Criteria

Any potential subject who meets any of the following criteria will be excluded from participating in the study.
1. Subject has a diagnosis of primary amyloidosis, monoclonal gammopathy of undetermined significance, or smoldering multiple myeloma. Monoclonal gammopathy of undetermined significance is defined by presence of serum M-protein <3 g/dL; absence of lytic bone lesions, anemia, hypercalcemia, and renal insufficiency related to the M-protein; and (if determined) proportion of plasma cells in the bone marrow of 10% or less (Kyle et al., Mayo Clin Proc 78:21-33, 2003). Smoldering multiple myeloma is defined as asymptomatic multiple myeloma with absence of related organ or tissue impairment end organ damage (Kyle et al., Mayo Clin Proc 78:21-33, 2003; Kyle et al., N Engl J Med 356:2582-2590, 2007)
2. Subject has a diagnosis of Waldenstrom's disease, or other conditions in which IgM M-protein is present in the absence of a clonal plasma cell infiltration with lytic bone lesions.
3. Subject has prior or current systemic therapy or SCT for multiple myeloma, with the exception of an emergency use of a short course (equivalent of dexamethasone 40 mg/day for 4 days) of corticosteroids before treatment.
4. Subject has a history of malignancy (other than multiple myeloma) within 5 years before the date of randomization (exceptions are squamous and basal cell carcinomas of the skin and carcinoma in situ of the cervix, or malignancy that in the opinion of the investigator, with concurrence with the sponsor's medical monitor, is considered cured with minimal risk of recurrence within 5 years).
5. Subject has had radiation therapy within 14 days of randomization.
6. Subject has had plasmapheresis within 28 days of randomization.
7. Subject is exhibiting clinical signs of meningeal involvement of multiple myeloma.
8. 8.1a) Subject has known chronic obstructive pulmonary disease (COPD) with a Forced Expiratory Volume in 1 second (FEV1)<50% of predicted normal. Note that FEV1 testing is required for subjects suspected of having COPD and subjects must be excluded if FEV1<50% of predicted normal
   8.1b) Subject has had known moderate or severe persistent asthma within the last 2 years or currently has uncontrolled asthma of any classification. (Note that subjects who currently have controlled intermittent asthma or controlled mild persistent asthma are allowed in the study).
9. Subject is known to be seropositive for human immunodeficiency virus (HIV) or hepatitis B (defined by a positive test for hepatitis B surface antigen [HBsAg] or antibodies to hepatitis B surface and core antigens [anti-HBs and anti-HBc, respectively]) or hepatitis C (anti-HCV antibody positive or HCV-RNA quantitation positive).
10. Subject has any concurrent medical or psychiatric condition or disease (eg, active systemic infection, uncontrolled diabetes, acute diffuse infiltrative pulmonary disease) that is likely to interfere with the study procedures or results, or that in the opinion of the investigator, would constitute a hazard for participating in this study.
11. Subject has clinically significant cardiac disease, including:
   myocardial infarction within 1 year before randomization, or an unstable or uncontrolled disease/condition related to or affecting cardiac function (eg, unstable angina, congestive heart failure, New York Heart Association Class III-IV)
   uncontrolled cardiac arrhythmia (National Cancer Institute Common Terminology Criteria for Adverse Events [NCI CTCAE] Version 4 Grade >3) or clinically significant ECG abnormalities
   screening 12-lead ECG showing a baseline QT interval as corrected by Fridericia's formula (QTcF) >470 msec
12. Subject has known allergies, hypersensitivity, or intolerance to corticosteroids, monoclonal antibodies or human proteins, lenalidomide, or their excipients (refer to respective package inserts or Investigator's Brochure), or known sensitivity to mammalian-derived products.
13. Subject has plasma cell leukemia (according to World Health Organization [WHO] criterion: >20% of cells in the peripheral blood with an absolute plasma cell count of more than 2×10⁹/L) or POEMS syndrome (polyneuropathy, organomegaly, endocrinopathy, monoclonal protein, and skin changes).
14. Subject is known or suspected of not being able to comply with the study protocol (e.g., because of alcoholism, drug dependency, or psychological disorder). Subject has any condition for which, in the opinion of the investigator, participation would not be in the best interest of the subject (e.g., compromise the well-being) or that could prevent, limit, or confound the protocol-specified assessments. Subject is taking any prohibited medications.
15. Subject is a woman who is pregnant, or breast-feeding, or planning to become pregnant while enrolled in this study, within 4 weeks after the last dose of lenalidomide, or within 3 months after the last dose of daratumumab)(DARZALEX®. Or, subject is a man who plans to father a child while enrolled in this study, within 4 weeks after the last dose of lenalidomide, or within 3 months after the last dose of daratumumab)(DARZALEX®.
16. Subject has had major surgery within 2 weeks before randomization or has not fully recovered from surgery, or has surgery planned during the time the subject is expected to participate in the study. Kyphoplasty or vertebroplasty is not considered major surgery.
17. Subject has received an investigational drug (including investigational vaccines) or used an invasive investigational medical device within 4 weeks before randomization or is currently enrolled in an interventional investigational study.
18. Subject has contraindications to required prophylaxis for deep vein thrombosis and pulmonary embolism.
19. Incidence of gastrointestinal disease that may significantly alter the absorption of oral drugs.

### Prevention of Infusion Reactions

Preinfusion medications for subjects receiving daratumumab (DARZALEX®) will be administered as follows. On daratumumab (DARZALEX®) infusion days, subjects will receive the following medications prior to infusion:
Acetaminophen (paracetamol) 650-1000 mg IV or orally (PO) approximately 1 hour or less prior to daratumumab)(DARZALEX® infusion
An antihistamine (diphenhydramine 25-50 mg IV or PO, or equivalent but avoid IV use of promethazine) approximately 1 hour prior to infusion after Cycle 6, if a subject has not developed an infusion-related reaction and is intolerant to antihistamines, modifications are acceptable as per investigator discretion.
Dexamethasone 40 mg IV (preferred) or PO, approximately 1 hour or less prior to daratumumab (DARZALEX®) infusion. For subjects older than 75 years or underweight (body mass index [BMI]<18.5), dexamethasone 20 mg may be administered as appropriate. An equivalent intermediate-acting or long-acting corticosteroid may substitute. On days when subjects receive this dose of dexamethasone in the clinic, dexamethasone will not be self-administered at home. If weekly dexamethasone dosing has been reduced below 10 mg due to adverse events during study, a minimum of dexamethasone 10 mg IV should continue to be administered prior to daratumumab (DARZALEX®) infusions.
   If necessary, all PO preinfusion medications may be administered outside of the clinic on the day of the infusion, provided they are taken within 3 hours before the infusion.

### Postinfusion Medication

For subjects with higher risk of respiratory complications (i.e., subjects with mild asthma, or subjects with COPD who have a FEV1<80%), the following postinfusion medications should be considered:
Antihistamine (diphenhydramine or equivalent)
Short-acting β₂ adrenergic receptor agonist such as salbutamol aerosol
Control medications for lung disease (e.g., inhaled corticosteroids±long-acting β₂ adrenergic receptor agonists for subjects with asthma; long-acting bronchodilators such as tiotropium or salmeterol±inhaled corticosteroids for subjects with COPD)

### Lenalidomide Dose Reductions

Dose adjustments of lenalidomide will follow the approved labeling as follows:
Starting dose: 25 mg
Dose level 1: 15 mg
Dose level 2: 10 mg
Dose level 3: 5 mg

Dose adjustments should be based on the highest grade of toxicity that is ascribed to lenalidomide. After initiation of lenalidomide, subsequent lenalidomide dose adjustment is based on individual subject treatment tolerance. If the investigator determines that an adverse event may be related to lenalidomide, dose adjustment can be done even if not specified in this protocol.

### Response Categories

Disease evaluations must be performed every 28 days for the first 2 years and then every 8 weeks until disease progression. A window of ±7 days is allowed. If treatment has been delayed for any reason, the disease evaluations must be performed according to schedule, regardless of any changes to the dosing regimen.

Disease evaluations will be performed by a central laboratory (unless otherwise specified). This study will use the IMWG consensus recommendations for multiple myeloma treatment response criteria (Durie et al., Leukemia, 20:1467-7143, 2006, Rajkumar et al., Blood, 117:4691-4695, 2011) presented in Table 1. For quantitative immunoglobulin, M-protein, and immunofixation measurements in serum and 24 hour urine, the investigator will use results provided by the central laboratory. Subjects with positive serum IFE and confirmed daratumumab (DARZALEX®) IFE interference, that meet all other clinical criteria for complete response or stringent complete response, will be considered CRIsCR.

Disease progression must be consistently documented across clinical study sites using the criteria in Table 1. For patients with measurable disease by SPEP or UPEP at baseline, increases in serum free light chains (FLC) or the FLC ratio alone do not meet criteria for progressive disease.

### Example 2: A Phase 3 Study Comparing Daratumumab (DARZALEX®), Lenalidomide, and Dexamethasone (DRd) Vs Lenalidomide and Dexamethasone (Rd) in Subjects with Previously Untreated Multiple Myeloma Who are Ineligible for High Dose Chemotherapy (HDC) and Autologous Stem Cell Transplant (ASCT)—Interim Analysis at Median Follow-Up of 28 Months

737 patients with newly diagnosed myeloma ineligible for HDC and ASCT were randomly assigned to receive lenalidomide and dexamethasone, either alone (control group) or with daratumumab (DARZALEX®) (daratumumab (DARZALEX®) group), the treatments continued until disease progression or unacceptable toxicity. The primary endpoint was progression-free survival. The study protocol is described in Example 1.

After median follow-up of 28 months, median progression-free survival was not reached in the daratumumab (DARZALEX®) group versus 31.9 months in the control group (hazard ratio, 0.56; 95% confidence interval, 0.43-0.73; P<0.0001). Rates of complete response or better were 47.6% versus 24.9% in the daratumumab (DARZALEX®) and control groups, respectively (P<0.0001). In the daratumumab (DARZALEX®) group, 24.2% of patients were minimal residual disease-negative (threshold of 1 tumor cell per 10⁵ white cells) versus 7.3% of patients in the control group (P<0.0001). The most common (>10%) grade 3/4 adverse events in the daratumumab (DARZALEX®) versus control groups were neutropenia (50.0% vs. 35.3%), lymphopenia (15.1% vs. 10.7%), pneumonia (13.7% vs. 7.9%), anemia (11.8% vs. 19.7%), and leukopenia (11.0% vs. 4.9%).

Daratumumab (DARZALEX®) plus lenalidomide and dexamethasone significantly decreased the risk of disease progression or death versus lenalidomide and dexamethasone alone in patients with newly diagnosed myeloma not eligible for autologous stem-cell transplantation. Higher rates of neutropenia and pneumonia were observed in the daratumumab (DARZALEX®) group.

In this randomized, open-label, active-controlled, multicenter phase 3 trial, patients were enrolled between March 2015 and January 2017 at sites located in 14 countries across North America, Europe, the Middle East, and the Asia-Pacific region. Independent ethics or institutional review boards at each site approved the protocol. The trial was conducted in accordance with the principles of the Declaration of Helsinki and the International Conference on Harmonisation—Good Clinical Practice guidelines. All patients provided written informed consent. Janssen Research & Development, LLC sponsored this trial and compiled/maintained the data.

### Patients

Eligible patients had documented newly diagnosed myeloma (Rajkumar et al., Lancet Oncol 15:e538-e548, 2014) Eastern Cooperative Oncology Group performance status ≤2, and were ineligible for high-dose chemotherapy with stem-cell transplantation due to age (65 years or older) or comorbidities. Patients had hemoglobin ≥7.5 g/dL, absolute neutrophil count ≥1.0×10⁹/L, platelet count ≥70×10⁹/L (≥50×10⁹/L if ≥50% of bone marrow nucleated cells were plasma cells), aspartate aminotransferase and alanine aminotransferase ≤2.5 times the upper limit of normal, total bilirubin ≤2.0 times the upper limit of normal, creatinine clearance ≥30 mL/minute, and corrected serum calcium ≤14 mg/dL.

### Trial Treatments

Patients were randomized using an interactive web response system (1:1 ratio) to daratumumab (DARZALEX®) in combination with lenalidomide and dexamethasone (daratumumab (DARZALEX®) group) or lenalidomide and dexamethasone alone (control group). Patients were stratified by International Staging System (ISS; I vs. II vs. III), region (North America vs. Other), and age (<75 years vs. >75 years).

During each 28-day cycle, all patients received oral lenalidomide (25 mg, Days 1-21) and oral dexamethasone (40 mg, Days 1, 8, 15, and 22) until disease progression or unacceptable toxicity. For patients older than 75 years of age or with body mass index less than 18.5 kg/m², dexamethasone was administered at a dose of 20 mg once weekly. Patients in the daratumumab (DARZALEX®) group also received intravenous daratumumab (DARZALEX®) 16 mg/kg once weekly in Cycles 1-2, every 2 weeks in Cycles 3-6, and every 4 weeks thereafter. Pre-infusion medications were administered to manage infusion reactions.

### Endpoints and Assessments

The primary endpoint was progression-free survival (time from date of randomization to either disease progression or death). Secondary efficacy endpoints were time to progression, complete response rate, stringent complete response rate, minimal residual disease-negativity rate (at a threshold of 1 tumor cell per 10⁵ white cells), the time from randomization to progression on next line of therapy or death, whichever comes first (progression-free survival 2), overall survival, overall response rate, the proportion of patients achieving very good partial response or better, time to response and duration of response, efficacy in high-risk molecular subgroups, and safety. Progressive disease was determined according to the International Myeloma Working Group criteria (Rajkumar et al., Blood 117:4691-4695, 2011; Durie et al., Leukemia 20:1467-1473, 2006).

A central laboratory performed disease evaluations (serum and 24-hour urine samples) every 28 days for 2 years, and then every 8 weeks until disease progression. For patients with positive serum immunofixation and daratumumab (DARZALEX®) interference, complete responses were confirmed using reflex assays (McCudden et al., Clin Chem Lab Med 54:1095-1104, 2016). Minimal residual disease was evaluated by next-generation sequencing assays (clonoSEQ® version 2.0; Adaptive Biotechnologies) on bone marrow aspirates collected at baseline, at time of suspected complete or stringent complete response (undetectable M-protein on two consecutive serum and urine electrophoresis tests), and at 12, 18, 24, and 30 months post-first dose in patients who achieved a complete response or better.

Safety analyses included adverse event assessment graded in severity according to NCI-CTCAE version 4, electrocardiograms, clinical laboratory testing, physical examinations, and vital signs.

### Statistical Analysis

The primary analysis population included all randomized patients in the intent-to-treat population. The safety population included patients who received any dose of trial treatment. A stratified log-rank test was used for the primary endpoint of progression-free survival. Treatment effect and 95% confidence intervals (CIs) were estimated using a stratified Cox regression model with treatment as the sole explanatory variable. Other time-to-event efficacy endpoints were analyzed similarly. Response to trial treatment and progressive disease was evaluated by a previously described validated computer algorithm (Dimopoulos et al., N Engl J Med 375:1319-1331 2016; Palumbo et al., N Engl J Med 375:754-766, 2016). Continuous, categorical, and time-to-event variables were summarized using descriptive statistics, frequency tables, and the Kaplan-Meier method, respectively. Binary endpoints were analyzed using the stratified Cochran-Mantel-Haenszel test. If the primary endpoint was statistically significant, the following secondary endpoints, as ordered here, were sequentially tested, each with an overall two-sided alpha of 0.05: rates of complete response or better, very good partial response or better, and negative status for minimal residual disease, overall response rate, and overall survival.

Two planned interim analyses were conducted. The first evaluated safety after 100 patients had received at least 8 weeks of treatment or had discontinued treatment. The second, reported here, assessed safety and efficacy after 240 progression-free survival events (62% of 390 planned progression-free survival events for primary analysis). The trial will end when 330 deaths are reported.

A sample size of 730 patients was estimated to provide 80% power to detect a reduction in the risk of progression or death by 25% in the daratumumab (DARZALEX®) group versus control group with a log-rank test with a two-sided alpha level of 0.05.

### Results

### Patients and Treatment

Of 737 enrolled patients, 368 and 369 were randomized to the daratumumab (DARZALEX®) and control groups, respectively. Baseline demographic and clinical characteristics were well balanced (Table 2). The median age was 73.0 years (range, 45 to 90), and 14.3% of patients had a high-risk cytogenetic profile. The median time since diagnosis was 0.9 months (range, 0 to 14.5).

Among randomized patients, 729 patients (364 in the daratumumab (DARZALEX®) group and 365 in the control group) received at least one dose of trial treatment. At the clinical cutoff date (Sep. 24, 2018), 118 patients (32.4%) in the daratumumab (DARZALEX®) group and 207 patients (56.7%) in the control group had discontinued treatment, most commonly due to progressive disease (14.6% vs. 23.8%) and adverse events (7.4% vs. 16.2%).

TABLE 2

| | Daratumumab | |
|---|---|---|
| | (DARZALEX ®) Group | Control Group |
| Characteristic | (N = 368) | (N = 369) |
| Age | | |
| Median (range)-years (yr) | 73.0 | 74.0 |
| | (50-90) | (45-89) |
| Distribution-no. (%) | | |
| <65 yr | 4 (1.1) | 4 (1.1) |
| 65-<70 yr | 74 (20.1) | 73 (19.8) |
| 70-<75 yr | 130 (35.3) | 131 (35.5) |
| ≥75 yr | 160 (43.5) | 161 (43.6) |
| ECOG performance | | |
| status-no. (%)^{†} | | |
| 0 | 127 (34.5) | 123 (33.3) |
| 1 | 178 (48.4) | 187 (50.7) |
| 2^{‡} | 63 (17.1) | 59 (16.0) |
| ISS disease | | |
| staging-no. (%)^{§} | | |
| I | 98 (26.6) | 103 (27.9) |
| II | 163 (44.3) | 156 (42.3) |
| III | 107 (29.1) | 110 (29.8) |
| Type of measurable | | |
| disease-no. (%) | | |
| IgG | 225 (61.1) | 231 (62.6) |
| IgA | 65 (17.7) | 66 (17.9) |
| Others^{¶} | 9 (2.4) | 10 (2.7) |
| Detected in urine only | 40 (10.9) | 34 (9.2) |
| Detected as serum free | 29 (7.9) | 28 (7.6) |
| light-chains only | | |
| Cytogenetic profile- | | |
| no./total no. (%)^{#} | | |
| Standard risk | 271/319 | 279/323 |
| | (85.0) | (86.4) |
| High risk | 48/319 | 44/323 |
| | (15.0) | (13.6) |
| Median time since | 0.95 | 0.89 |
| initial diagnosis of | (0.1-13.3) | (0-14.5) |
| multiple myeloma | | |
| (range)-months | | |

*The intention-to-treat population included all randomized patients. Post hoc analyses showed no significant differences between characteristics evaluated at baseline for the two groups.
^{†}Eastern Cooperative Oncology Group (ECOG) performance status is scored on a scale from 0 to 5, with 0 indicating no symptoms and higher scores indicating increasing disability.
^{‡}Two patients had an ECOG performance status of greater than 2 (one patient with ECOG performance status of 3; another patient with ECOG performance status of 4).
^{§}The International Staging System (ISS) disease stage is derived on the basis of the combination of serum β₂-microglobulin and albumin levels. Higher stages indicate more severe disease.
^{¶}Includes IgD, IgE, IgM, and biclonal.
^{#}Cytogenetic risk based on fluorescence in situ hybridization or karyotype testing; high cytogenetic risk patients had at least one high-risk abnormality (t[4;14], t[14;16], del17p).

The median duration of treatment was 25.3 months (range: 0.1 to 40.4) in the daratumumab (DARZALEX®) group and 21.3 months (range: 0.03 to 40.6) in the control group, and the median number of cycles received was 27 (range: 1 to 44) versus 22 (range: 1 to 43). The median relative dose intensity (the ratio of administered to planned doses) of daratumumab (DARZALEX®) was 98.4%. The median relative dose intensity of lenalidomide was 76.2% in the daratumumab (DARZALEX®) group and 91.4% in the control group; a higher rate of lenalidomide dose modifications due to treatment-emergent adverse events was reported in the daratumumab (DARZALEX®) versus control group, including dose discontinuations (20.9% vs. 17.0%, respectively) or dose delays, reductions, re-escalations, or skipping (combined: 77.5% vs. 64.7%, respectively). The median relative dose intensity of dexamethasone was 84.2% in the daratumumab) (DARZALEX® group and 90.7% in the control group.

### Efficacy

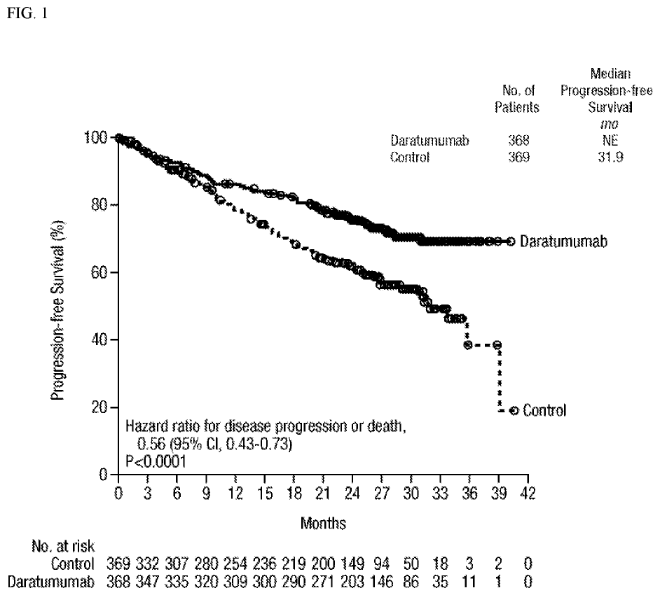

At a median duration of follow-up of 28.0 months (range: 0 to 41.4), a total of 240 events of disease progression or death (in 97 patients [26.4%] in the daratumumab (DARZALEX®) group vs. 143 [38.8%] in the control group) had occurred. The hazard ratio for disease progression or death in the daratumumab (DARZALEX®) group versus the control group was 0.56 (95% CI, 0.43 to 0.73; P<0.0001) (FIG. 1). The Kaplan-Meier estimate of the 30-month rate of progression-free survival was 70.6% (95% CI, 65.0 to 75.4) in the daratumumab (DARZALEX®) group and 55.6% (95% CI, 49.5 to 61.3) in the control group. The median progression-free survival was not reached (95% CI, could not be estimated) in the daratumumab (DARZALEX®) group versus 31.9 months (95% CI, 28.9 to could not be estimated) in the control group (P<0.0001). In the time-to-event analysis of disease progression, 179 events (in 66 patients [17.9%] in the daratumumab)(DARZALEX® group versus 113 [30.6%] patients in the control group) were observed, with median time to progression not being reached in the daratumumab)(DARZALEX® group compared with 35.8 months (95% CI, 31.4 to could not be estimated) in the control group (hazard ratio, 0.47; 95% CI, 0.35 to 0.64; P<0.0001).

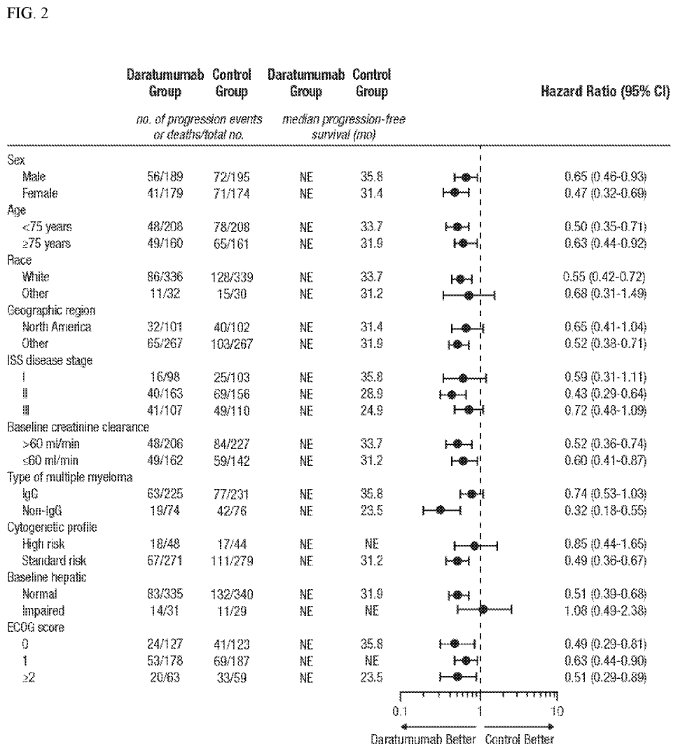

Prespecified subgroup analyses of progression-free survival confirmed the superiority of the daratumumab (DARZALEX®) group over the control group across all subgroups, except those patients with hepatic impairment (FIG. 2). The progression-free survival benefit was maintained in patients 75 years of age or older (hazard ratio, 0.63; 95% CI, 0.44 to 0.92) and among patients with historically poor prognosis, including those with a high-risk cytogenetic profile (hazard ratio, 0.85; 95% CI, 0.44 to 1.65) and ISS disease stage III (hazard ratio, 0.72; 95% CI, 0.48 to 1.09). Although the hazard ratio for disease progression or death was lower for patients with a standard-risk cytogenetic profile (hazard ratio, 0.49) than for those with a high-risk cytogenetic profile, the results favored the daratumumab (DARZALEX®) group in both subpopulations. The small number of patients with a high-risk cytogenetic profile limits the interpretation of these findings.

In the intention-to-treat population (e.g., all subjects who were enrolled and randomly allocated to treatment), patients in the daratumumab (DARZALEX®) group achieved significantly higher rates of complete response or better (47.6% vs. 24.9%, P<0.0001) and of very good partial response or better (79.3% vs. 53.1%, P<0.0001) compared with the control group (Table 3). The overall response rate was 92.9% in the daratumumab)(DARZALEX® group and 81.3% in the control group (P<0.0001).

The higher rates of deeper responses in the daratumumab (DARZALEX®) group were evidenced by a negative status for minimal residual disease (at a threshold of 1 tumor cell per 10⁵ white cells) that was more than 3 times as high in the daratumumab (DARZALEX®) group versus the control group (24.2% vs. 7.3%, P<0.0001) (Table 3). Patients with negative status for minimal residual disease demonstrated longer progression-free survival compared with those with positive status, regardless of trial treatment. All patients who achieved negative status for minimal residual disease had achieved complete response or better.

A total of 138 deaths occurred (62 in the daratumumab (DARZALEX®) group vs. 76 in the control group). The median overall survival was not reached in either treatment group (hazard ratio, 0.78; 95% CI, 0.56 to 1.10; P=0.1528), and long-term follow-up is ongoing.

The median duration of response was not reached (95% CI, could not be estimated) in the daratumumab (DARZALEX®) group versus 34.7 months (95% CI, 30.8 to could not be estimated) in the control group. The median time to first response among responders was 1.05 months in both groups and the median time to complete response or better was 10.4 months in the daratumumab (DARZALEX®) group and 11.2 months in the control group. Minimal residual disease-negative events accumulated faster in the daratumumab (DARZALEX®) arm.

The progression-free survival benefit observed with daratumumab (DARZALEX®) was maintained with the next line of therapy as demonstrated by a longer duration of progression-free survival 2 in the daratumumab (DARZALEX®) ° group than in the control group (median not reached in either treatment group; hazard ratio, 0.70; 95% CI, 0.51 to 0.96; P=0.0278); the 36-month rate of progression-free survival 2 was 77.1% (95% CI, 70.6 to 82.3) in the daratumumab)(DARZALEX® group and 65.2% (95% CI, 54.2 to 74.1) in the control group. A total of 155 events of progression or death were observed while patients were receiving the next line of therapy (68 patients in the daratumumab (DARZALEX®) group and 87 patients in the control group).

TABLE 3

| | Daratumumab | | |
|---|---|---|---|
| | (DARZALEX ®) | Control Group | |
| Response Catergory | Group (N = 368) | (N = 369) | P Value |
| Overall response | | | |
| No. with response | 342 | 300 | |
| Rate-0 % (95% CI) | 92.9 | 81.3 | <0.0001^{‡} |
| | (89.8-95.3) | (76.9-85.1) | |
| Best overall response-no. (%) | | | |
| Complete response or better | 175 (47.6) | 92 (24.9) | <0.0001^{‡} |
| Stringent complete response^{§} | 112 (30.4) | 46 (12.5) | |
| Complete response | 63 (17.1) | 46 (12.5) | |
| Very good partial response or better | 292 (79.3) | 196 (53.1) | <0.0001^{‡} |
| Very good partial response | 117 (31.8) | 104 (28.2) | |
| Partial response | 50 (13.6) | 104 (28.2) | |
| Stable disease | 11 (3.0) | 56 (15.2) | |
| Progressive disease | 1 (0.3) | 0 | |
| Response could not be evaluated | 14 (3.8) | 13 (3.5) | |
| Negative status for minimal | | | |
| residual disease-no. (%)^{¶} | 89 (24.2) | 27 (7.3) | <0.0001^{#} |

*Response was assessed in the intention-to-treat population on the basis of International Myeloma Working Group recommendations (details on the criteria for disease responses are provided in the protocol).
^{†}The following secondary endpoints were sequentially tested, each with an overall two-sided alpha of 0.05, by utilizing a hierarchical testing approach: rate of complete response or better, rate of very good partial response or better, rate of negative status for minimal residual disease, overall response rate.
^{‡}P value was calculated using the Cochran-Mantel-Haenszel chi-square test.
^{§}Criteria for a stringent complete response include the criteria for a complete response plus a normal free light-chain ratio and absence of clonal plasma cells, as assessed by immunofluorescence or immunohistochemical analysis or by two-color to four-color flow cytometry.
^{¶}Minimal residual disease-negativity status, using a sensitivity threshold of 1 tumor cell per 10⁵ white cells, is based on a post-randomization assessment of bone marrow samples using a validated next-generation sequencing assay (clonoSEQ ® Assay version 2.0, Adaptive Biotechnologies) in accordance with the minimal residual disease assessment guidelines established by the International Myeloma Working Group.²⁵
^{#}P value was calculated using the Fisher's exact test.

### Safety

Table 4 summarizes the most common adverse events of any grade during treatment (in more than 30% of patients in either group) or adverse events of grade 3 or 4 (in more than 10% of patients in either group) for the safety population; the most common adverse events of grade 3 or 4 were neutropenia (50.0% vs. 35.3%, respectively), lymphopenia (15.1% vs. 10.7%, respectively), pneumonia (13.7% vs. 7.9%, respectively), anemia (11.8% vs. 19.7%, respectively), and leukopenia (11.0% vs. 4.9%, respectively). The rate of any-grade infections was 86.3% in the daratumumab (DARZALEX®) group and 73.4% in the control group; rates of grade 3 or 4 infections were 32.1% and 23.3%, respectively.

Serious adverse events were reported in 62.9% of patients in the daratumumab (DARZALEX®) group and 62.7% of patients in the control group, among which pneumonia was the most common, occurring in 13.2% and 7.4% of patients, respectively. The percentage of patients with adverse events leading to discontinuation of trial treatment was 7.1% in the daratumumab (DARZALEX®) group and 15.9% in the control group. Discontinuation of trial treatment due to infections was 0.5% in the daratumumab (DARZALEX®) group and 1.4% in the control group; no patients in the daratumumab (DARZALEX®) group discontinued treatment due to neutropenia compared with 1 (0.3%) patient in the control group.

Adverse events leading to death were observed in 25 patients (6.9%) in the daratumumab (DARZALEX®) group and 23 patients (6.3%) in the control group; the most common was pneumonia, occurring in 0.5% and 0.8% of patients, respectively. Invasive second primary malignancies were reported in 12 (3.3%) patients in the daratumumab (DARZALEX®) group (2.7% solid tumors; 0.5% hematologic malignancies) and 13 (3.6%) patients in the control group (3.0% solid; 0.5% hematologic).

Daratumumab (DARZALEX®)-associated infusion-related reactions were reported in 40.9% of patients; 2.7% were grade 3 or 4 events (with one patient reporting grade 4 hypertension), and no grade 5 events were reported. Infusion-related reactions usually occurred during the first dose (in 98.0% of patients with infusion reactions), and only one patient discontinued daratumumab (DARZALEX®) treatment due to an infusion-related reaction (grade 4 hypertension).

TABLE 4

| | Daratumumab | | | |
|---|---|---|---|---|
| | (DARZALEX) | | Control Group | |
| | Group (N = 364) | | (N = 365) | |
| Event | Any Grade | Grade 3 or 4 | Any Grade | Grade 3 or 4 |
| | number of patients (percent) | | | |
| Hematologic adverse | | | | |
| events | | | | |
| Neutropenia | 207 (56.9) | 182 (50.0) | 154 (42.2) | 129 (35.3) |
| Anemia | 126 (34.6) | 43 (11.8) | 138 (37.8) | 2 (19.7) |
| Leukopenia | 68 (18.7) | 40 (11.0) | 34 (9.3) | 18 (4.9) |
| Lymphopenia | 66 (18.1) | 55 (15.1) | 45 (12.3) | 39 (10.7) |
| Nonhematologic adverse | | | | |
| events | | | | |
| Infections | 314 (86.3) | 117 (32.1) | 268 (73.4) | 85 (23.3) |
| Pneumonia | 82 (22.5) | 50 (13.7) | 46 (12.6) | 29 (7.9) |
| Diarrhea | 207 (56.9) | 24 (6.6) | 168 (46.0) | 15 (4.1) |
| Constipation | 149 (40.9) | 6 (1.6) | 130 (35.6) | 1 (0.3) |
| Fatigue | 147 (40.4) | 29 (8.0) | 104 (28.5) | 14 (3.8) |
| Peripheral edema | 140 (38.5) | 7 (1.9) | 107 (29.3) | 2 (0.5) |
| Back pain | 123 (33.8) | 11 (3.0) | 96 (26.3) | 11 (3.0) |
| Asthenia | 117 (32.1) | 16 (4.4) | 90 (24.7) | 13 (3.6) |
| Nausea | 115 (31.6) | 5 (1.4) | 84 (23.0) | 2 (0.5) |
| Second primary | 32 (8.8) | NA | 26 (7.1) | NA |
| malignancy^{†} | | | | |
| Invasive second | 12 (3.3) | NA | 13 (3.6) | NA |
| primary malignancy | | | | |
| Any infusion-related | 149 (40.9) | 10 (2.7) | NA | NA |
| reaction | | | | |

*All patients who received at least one dose of trial treatment were included in the safety population. Adverse events of any grade that were reported in more than 30% of patients in either treatment group or grade 3 or 4 adverse events that were reported in more than 10% of patients in either treatment group are listed. NA denotes not applicable.
^{†}The presence of a second primary malignancy was prespecified in the statistical analysis plan as an adverse event of clinical interest.

### Example 3: Impact of Age on the Efficacy and Safety of Daratumumab (DARZALEX®) in Combination with Lenalidomide and Dexamethasone (DRd) in Patients with Transplant-Ineligible Newly Diagnosed Multiple Myeloma (NDMM): MAIA

DRd significantly reduced the risk of progression or death by 44% in transplant-ineligible NDMM pts vs Rd in the primary analysis of the phase 3 MAIA study (Example 2). To examine the impact of age on the efficacy and safety of D-Rd vs Rd in this patient population, a subgroup analysis was conducted within patients <75 and >75 y of age.

Methods:

Transplant-ineligible NDMM patients were randomized 1:1 to Rd±DARA; stratification was based on age (<75 vs >75 years), ISS (I, II, III), and region (North America vs Other). In standard Rd dosing, patients received 28-day cycles of lenalidomide 25 mg PO QD on Days 1-21 and dexamethasone 40 mg PO on Days 1, 8, 15 and 22 until progression. A portion of patients received 10 mg lenalidomide and 20 mg dexamethasone at the beginning of the treatment. In the DRd arm, patients received daratumumab (DARZALEX®) 16 mg/kg IV QW for Cycles 1-2, Q2W for Cycles 3-6, and Q4W thereafter until progression. PFS was the primary endpoint.

Results:

Among 737 randomized patients (D-Rd, n=368; Rd, n=369), 321 (44%) were >75 y of age. A higher proportion of patients in the D-Rd arm received a lower starting dose of lenalidomide (10 mg) compared with the Rd arm (30.8% vs 22.7%), and a lower relative median dose intensity for lenalidomide (<75 y: 79% vs 93%; >75 y: 66% vs 89%). After median follow-up of 28 months, significant PFS benefit of D-Rd vs Rd was maintained in both <75 and >75 y subgroups (<75: median not reached [NR] vs 33.7 mo; HR 0.50; 95% CI 0.35-0.71; >75 y: median NR vs 31.9 months; HR 0.63; 95% CI 0.44-0.92. Overall response rate (<75:95% vs 82%; >75 y: 90% vs 81%), rate of complete response or better (<75:52% vs 25%; >75 y: 41% vs 25%), rate of very good partial response or better (<75:81% vs 53%; >75 y: 77% vs 53%), and minimal residual disease-negative rate (10⁻⁵ threshold; <75:28% vs 7%; >75 y: 19% vs 8%) remained higher with D-Rd vs Rd in both age subgroups. Most common (>10%; D-Rd/Rd) grade 3/4 TEAEs in <75 y patients were neutropenia (43%/31%), pneumonia (13%/6%), lymphopenia (12%/10%), leukopenia (10%/4%), and anemia (9%/18%). Most common (>10%; D-Rd/Rd) grade 3/4 TEAEs in >75 y patients were neutropenia (60%/41%), lymphopenia (19%/12%), anemia (16%/22%), pneumonia (15%/10%), leukopenia (12%/6%), and thrombocytopenia (8%/11%). Fewer patients receiving D-Rd vs Rd discontinued treatment due to TEAEs (<75 y: 5% vs 12%; >75 y: 10% vs 21%); discontinuation rates due to infections for D-Rd vs Rd were low in both age groups (<75 y: 1% vs 1%; >75 y: 0% vs 2%). A higher proportion of >75 y patients discontinued lenalidomide due to TEAEs compared with <75 y patients (>75 y: 29% vs 22%; <75 y: 15% vs 13%).

Conclusions:

DRd patients received less lenalidomide than the Rd group regardless of age. Efficacy of DRd in <75 y and >75 y pts were consistent with the ITT population, and DRd demonstrated acceptable tolerability regardless of age. Together with the phase 3 ALCYONE study, these studies confirm clinical benefit of daratumumab (DARZALEX®) plus standard-of-care in transplant-ineligible NDMM pts >75 y of age.

TABLE 5

| | <75 y | | ≥75 y | |
|---|---|---|---|---|
| | DRd | Rd | DRd | Rd |
| | (n = 208) | (n = 208) | (n = 160) | (n = 161) |
| PFS | | | | |
| Median, months | NR | 33.7 | NR | 31.9 |
| HR (95% CI) | 0.50 (0.35-0.71) | | 0.63 (0.44-0.92) | |
| 30-mo PFS, % | 75 | 58 | 66 | 52 |
| ORR, % | 95 | 82 | 90 | 81 |
| ≥CR, % | 52 | 25 | 41 | 25 |
| ≥VGPR, % | 81 | 53 | 77 | 53 |
| MRD-negative | 28 | 7 | 19 | 8 |
| rate, % (10⁻⁵⁾ | | | | |
| NR: not reached | | | | |

Sequence listing

| | Amino Acid Sequence | SEQ ID NO |
|---|---|---|
| HCDR1 | SFAMS | 1 |
| HCDR2 | AISGSGGGTYYADSVKG | 2 |
| HCDR3 | DKILWFGEPVFDY | 3 |
| LCDR1 | RASQSVSSYLA | 4 |
| LCDR2 | DASNRAT | 5 |
| LCDR3 | QQRSNWPPTF | 6 |
| VH | EVQLLESGGGLVQPGGSLRLSCAVSGFTFNSFAMSWVRQAP | 7 |
| | GKGLEWVSAISGSGGGTYYADSVKGRFTISRDNSKNTLYLQ | |
| | MNSLRAEDTAVYFCAKDKILWFGEPVFDYWGQGTLVTVSS | |
| VL | EIVLTQSPATLSLSPGERATLSCRASQSVSSYLAWYQQKPGQ | 8 |
| | APRLLIYDASNRATGIPARFSGSGSGTDFTLTISSLEPEDFAVY | |
| | YCQQRSNWPPTFGQGTKVEIK | |
| HC | EVQLLESGGGLVQPGGSLRLSCAVSGFTFNSFAMSWVRQAP | 9 |
| | GKGLEWVSAISGSGGGTYYADSVKGRFTISRDNSKNTLYLQ | |
| | MNSLRAEDTAVYFCAKDKILWFGEPVFDYWGQGTLVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKRVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| LC | EIVLTQSPATLSLSPGERATLSCRASQSVSSYLAWYQQKPGQ | 10 |
| | APRLLIYDASNRATGIPARFSGSGSGTDFTLTISSLEPEDFAVY | |
| | YCQQRSNWPPTFGQGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| IgG1 | ASTKGPSVFPLAPSSKSTSGGTAALGCLVKDYFPEPVTVSWN | 11 |
| constant | SGALTSGVHTFPAVLQSSGLYSLSSVVTVPSSSLGTQTYICNV | |
| domain | NHKPSNTKVDKRVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |

What is claimed is:

1) A method of treating a subject with newly diagnosed multiple myeloma, comprising administering or providing for administration to the subject daratumumab, wherein daratumumab is administered as a combination therapy with lenalidomide and dexamethasone, and wherein the method achieves an improved clinical efficacy endpoint when compared to a clinical efficacy endpoint achieved if the subject were administered a combination of lenalidomide and dexamethasone.

2) The method of claim 1, wherein the improved clinical efficacy endpoint is an increased likelihood of achieving a complete response (CR) or better, an increased likelihood of achieving a very good partial response (VGPR) or better, an increased likelihood of achieving a negative status for minimal residual disease (MRD), a reduced risk of progression of multiple myeloma or death, a prolonged progression-free survival (PFS), or an increased likelihood of achieving a 30-month rate of progression-free survival, or any combination thereof

3) The method of claim 2, wherein the likelihood of achieving the CR or better is about 47% or higher.

4) The method of claim 3, wherein the likelihood of achieving the VGPR or better is about 79% or higher.

5) The method of claim 4, wherein the likelihood of achieving the negative status for MRD is about 24% or higher.

6) The method of claim 5, wherein the risk of progression of multiple myeloma or death is reduced by about 44%.

7) The method of claim 6, wherein the subject with newly diagnosed multiple myeloma is ineligible for HDC and ASCT.

8) The method of claim 7, wherein the combination therapy comprises about 16 mg/kg daratumumab, about 25 mg lenalidomide and between about 20 mg and about 40 mg dexamethasone.

9) The method of claim 8, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

10) The method of claim 9, wherein dexamethasone is administered as pre-medication on daratumumab administration days.

11) The method of claim 10, wherein daratumumab is administered intravenously, lenalidomide is administered orally and dexamethasone is administered intravenously or orally.

12) The method of claim 11, wherein lenalidomide, dexamethasone or both lenalidomide and dexamethasone are self-administered.

13) The method of claim 8, wherein daratumumab is provided for administration by a manufacturer of daratumumab in a single-dose vial comprising 100 mg daratumumab in 5 mL of solution or in a single-dose vial comprising 400 mg daratumumab in 20 mL of solution.

14) The method of claim 13, wherein each single-dose vial comprising 100 mg daratumumab in 5 mL of solution and each single-dose vial comprising 400 mg daratumumab in 20 mL of solution further comprises glacial acetic acid, mannitol, polysorbate 20, sodium acetate trihydrate and sodium chloride.

15) The method of claim 14, wherein each single-dose vial comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and each single-dose vial comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

16) The method of claim 13, wherein daratumumab is diluted into 0.9% sodium chloride prior to administration.

17) The method of claim 8, wherein information that the combination therapy comprising daratumumab, lenalidomide and dexamethasone achieves the improved clinical efficacy endpoint is provided on a daratumumab-containing drug product label.

18) The method of claim 17, wherein the daratumumab-containing drug product label includes information that a recommended dose of daratumumab is 16 mg/kg administered as an intravenous injection.

19) The method of claim 18, wherein the daratumumab-containing drug product label includes information that the recommended dosing schedule of daratumumab in combination with lenalidomide is once a week on weeks 1 to 8, once in two weeks on weeks 9-24 and thereafter once in four weeks.

20) The method of claim 19, wherein the daratumumab-containing drug product label includes information that the recommended dosing schedule of lenalidomide is 25 mg daily on days 1-21 of repeated 4 week cycles.

21) The method of claim 20, wherein the daratumumab-containing drug product label includes information that the recommended dosing schedule of dexamethasone is about 20 mg or about 40 mg per week.

22) The method of claim 17, wherein the daratumumab-containing drug product label includes data from an open-label, randomized active-controlled phase 3 study that compared treatment with daratumumab, lenalidomide and dexamethasone (DRd) to treatment with lenalidomide and dexamethasone (Rd) in subjects with newly diagnosed multiple myeloma who are ineligible for HDC and ASCT.

23) The method of claim 23, wherein the daratumumab-containing drug product label includes data that treatment with DRd resulted in about 44% reduction in the risk of multiple myeloma progression or death when compared to treatment with Rd.

24) The method of claim 24, wherein the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

25) The method of claim 25, wherein the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

26) The method of claim 26, wherein the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

27) The method of claim 27, wherein the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

28) The method of claim 28, wherein the daratumumab-containing drug product label includes data from a phase 3 active-controlled study that compared treatment with daratumumab, bortezomib and dexamethasone (DVd) to treatment with bortezomib and dexamethasone (Vd) in relapsed, refractory or relapsed and refractory multiple myeloma.

29) The method of claim 29, wherein the daratumumab-containing drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide bortezomib and dexamethasone.

30) The method of claim 30, wherein the daratumumab-containing drug product label includes information that side effects of daratumumab includes weakness, decreased appetite, bronchitis and lung infection.

31) The method of claim 31, wherein the daratumumab-containing drug product label includes information about approved indications, dosage and administrations, adverse reactions, drug product interactions, use in specific populations, clinical pharmacology, nonclinical toxicology, clinical studies and storage and handling of daratumumab, or any combination thereof.

32) The method of claim 32, wherein daratumumab is produced in a mammalian cell line.

33) The method of claim 33, wherein the mammalian cell line is a Chinese hamster ovary (CHO) cell line.

34) The method of claim 34, wherein the molecular weight of daratumumab is about 148 kDa.

35) The method of claim 8, wherein dexamethasone can be substituted for a dexamethasone equivalent, wherein the dexamethasone equivalent is methylprednisolone, prednisolone, prednisone or betamethasone, or any combination thereof.

36) A method of treating a subject with newly diagnosed multiple myeloma, comprising administering to the subject a combination therapy demonstrated to increase a likelihood of achieving a VGPR or better in subjects with multiple myeloma, wherein the combination therapy comprises daratumumab, lenalidomide and dexamethasone.

37) The method of claim 36, wherein the likelihood of achieving the VGPR or better is about 79% or higher.

38) The method of claim 37, wherein the subject with newly diagnosed multiple myeloma is ineligible for HDC and ASCT.

39) The method of claim 38, wherein the combination therapy comprises about 16 mg/kg daratumumab, about 25 mg lenalidomide and between about 20 mg and about 40 mg dexamethasone.

40) The method of claim 39, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

41) The method of claim 40, wherein dexamethasone is administered as pre-medication on daratumumab administration days.

42) The method of claim 41, wherein daratumumab is administered intravenously, lenalidomide is administered orally and dexamethasone is administered intravenously or orally.

43) The method of claim 42, wherein lenalidomide, dexamethasone or both lenalidomide and dexamethasone are self-administered.

44) A method of treating a subject with newly diagnosed multiple myeloma, comprising administering to the subject a combination therapy demonstrated to increase a likelihood of achieving a negative status for MRD in subjects with newly diagnosed multiple myeloma, wherein the combination therapy comprises daratumumab, lenalidomide and dexamethasone.

45) The method of claim 44, wherein the likelihood of achieving the negative status for MRD is about 24% or higher.

46) The method of claim 45, wherein the subject with newly diagnosed multiple myeloma is ineligible for HDC and ASCT.

47) The method of claim 46, wherein the combination therapy comprises about 16 mg/kg daratumumab, about 25 mg lenalidomide and between about 20 mg and about 40 mg dexamethasone.

48) The method of claim 47, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

49) The method of claim 48, wherein dexamethasone is administered as pre-medication on daratumumab administration days.

50) The method of claim 49, wherein daratumumab is administered intravenously, lenalidomide is administered orally and dexamethasone is administered intravenously or orally.

51) The method of claim 50, wherein lenalidomide, dexamethasone or both lenalidomide and dexamethasone are self-administered.

52) A method of treating a subject with newly diagnosed multiple myeloma, comprising administering to the subject a combination therapy demonstrated to increase a likelihood of achieving a CR or better in subjects with newly diagnosed multiple myeloma, wherein the combination therapy comprises daratumumab, lenalidomide and dexamethasone.

53) The method of claim 52, wherein the likelihood of achieving the CR or better is about 47% or higher.

54) The method of claim 53, wherein the subject with newly diagnosed multiple myeloma is ineligible for HDC and ASCT.

55) The method of claim 54, wherein the combination therapy comprises about 16 mg/kg daratumumab, about 25 mg lenalidomide and between about 20 mg and about 40 mg dexamethasone.

56) The method of claim 55, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

57) The method of claim 56, wherein dexamethasone is administered as pre-medication on daratumumab administration days.

58) The method of claim 57, wherein daratumumab is administered intravenously, lenalidomide is administered orally and dexamethasone is administered intravenously or orally.

59) The method of claim 58, wherein lenalidomide, dexamethasone or both lenalidomide and dexamethasone are self-administered.

60) A method of treating a subject with newly diagnosed multiple myeloma, comprising administering to the subject a combination therapy demonstrated to reduce a risk of progression of multiple myeloma or death in subjects with newly diagnosed multiple myeloma, wherein the combination therapy comprises daratumumab, lenalidomide and dexamethasone.

61) The method of claim 60, wherein the risk of progression of multiple myeloma or death is reduced by about 44%.

62) The method of claim 61, wherein the subject with newly diagnosed multiple myeloma is ineligible for HDC and ASCT.

63) The method of claim 62, wherein the combination therapy comprises about 16 mg/kg daratumumab, about 25 mg lenalidomide and between about 20 mg and about 40 mg dexamethasone.

64) The method of claim 63, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

65) The method of claim 64, wherein dexamethasone is administered as pre-medication on daratumumab administration days.

66) The method of claim 65, wherein daratumumab is administered intravenously, lenalidomide is administered orally and dexamethasone is administered intravenously or orally.

67) The method of claim 66, wherein lenalidomide, dexamethasone or both lenalidomide and dexamethasone are self-administered.

68) A method of treating a subject with newly diagnosed multiple myeloma, comprising:
a) providing a healthcare professional (HCP) daratumumab;
b) providing the HCP information that treating the subject with a combination therapy comprising daratumumab, lenalidomide and dexamethasone achieves an improved clinical efficacy endpoint when compared to a clinical efficacy endpoint achieved if the subject were treated with a combination of lenalidomide and dexamethasone; wherein performing the steps a) and b) results in the subject with newly diagnosed multiple myeloma to receive the combination therapy comprising daratumumab, lenalidomide and dexamethasone by the HCP or by self-administration as instructed by the HCP, thereby treating the subject having the newly diagnosed multiple myeloma.

69) The method of claim 68, wherein the improved clinical efficacy endpoint is an increased likelihood of achieving a CR or better, an increased likelihood of achieving a VGPR or better, an increased likelihood of achieving a negative status for MRD, a reduced risk of progression of multiple myeloma or death, a prolonged progression-free survival (PFS), or an increased likelihood of achieving a 30-month rate of progression-free survival, or any combination thereof.

70) The method of claim 69, wherein the likelihood of achieving the CR or better is about 47% or higher, the likelihood of achieving the VGPR or better is about 79% or higher, the likelihood of achieving the negative status for MRD is about 24% or higher or the risk of progression of multiple myeloma or death is reduced by about 44%, or any combination thereof.

71) The method of claim 70, wherein the subject with newly diagnosed multiple myeloma is ineligible for HDC and ASCT.

72) The method of claim 71, wherein the combination therapy comprises about 16 mg/kg daratumumab, about 25 mg lenalidomide and between about 20 mg and about 40 mg dexamethasone.

73) The method of claim 72, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

74) The method of claim 73, wherein dexamethasone is administered as pre-medication on daratumumab administration days.

75) The method of claim 74, wherein daratumumab is administered intravenously, lenalidomide is administered orally and dexamethasone is administered intravenously or orally.

76) The method of claim 75, wherein lenalidomide, dexamethasone or both lenalidomide and dexamethasone are self-administered.

77) The method of claim 72, wherein information that the combination therapy comprising daratumumab, lenalidomide and dexamethasone achieves the improved clinical efficacy endpoint is provided on a daratumumab-containing drug product label.

78) The method of claim 77, wherein the daratumumab-containing drug product label includes information that a recommended dose of daratumumab is 16 mg/kg administered as an intravenous injection.

79) The method of claim 78, wherein the daratumumab-containing drug product label includes information that the recommended dosing schedule of daratumumab in combination with lenalidomide is once a week on weeks 1 to 8, once in two weeks on weeks 9-24 and thereafter once in four weeks.

80) The method of claim 79, wherein the daratumumab-containing drug product label includes information that the recommended dosing schedule of lenalidomide is 25 mg daily on days 1-21 of repeated 4 week cycles.

81) The method of claim 80, wherein the daratumumab-containing drug product label includes information that the recommended dosing schedule of dexamethasone is about 20 mg or about 40 mg per week.

82) The method of claim 81, wherein the daratumumab-containing drug product label includes data from an open-label, randomized active-controlled phase 3 study that compared treatment with daratumumab, lenalidomide and dexamethasone (DRd) to treatment with lenalidomide and dexamethasone (Rd) in subjects with newly diagnosed multiple myeloma who are ineligible for HDC and ASCT.

83) The method of claim 82, wherein the daratumumab-containing drug product label includes data that treatment with DRd resulted in about 44% reduction in the risk of multiple myeloma progression or death when compared to treatment with Rd.

84) The method of claim 83, wherein the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

85) The method of claim 84, wherein the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

86) The method of claim 85, wherein the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

87) The method of claim 86, wherein the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

88) The method of claim 87, wherein the daratumumab-containing drug product label includes data from a phase 3 active-controlled study that compared treatment with daratumumab, bortezomib and dexamethasone (DVd) to treatment with bortezomib and dexamethasone (Vd) in relapsed, refractory or relapsed and refractory multiple myeloma.

89) The method of claim 88, wherein the daratumumab-containing drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide bortezomib and dexamethasone.

90) The method of claim 89, wherein the daratumumab-containing drug product label includes information that side effects of daratumumab includes weakness, decreased appetite, bronchitis and lung infection.

91) The method of claim 90, wherein the daratumumab-containing drug product label includes information about approved indications, dosage and administrations, adverse reactions, drug product interactions, use in specific populations, clinical pharmacology, nonclinical toxicology, clinical studies and storage and handling of daratumumab, or any combination thereof.

92) The method of claim 91, wherein daratumumab is produced in a mammalian cell line.

93) The method of claim 92, wherein the mammalian cell line is a Chinese hamster ovary (CHO) cell line.

94) The method of claim 93, wherein the molecular weight of daratumumab is about 148 kDa.

95) The method of claim 72, wherein dexamethasone can be substituted for a dexamethasone equivalent, wherein the dexamethasone equivalent is methylprednisolone, prednisolone, prednisone or betamethasone, or any combination thereof.

96) A method of providing daratumumab to a HCP for the HCP to treat a subject with newly diagnosed multiple myeloma with a combination therapy comprising daratumumab, lenalidomide and dexamethasone, wherein the treatment with the combination therapy comprising daratumumab, lenalidomide and dexamethasone achieves an improved clinical efficacy endpoint when compared to a clinical efficacy endpoint achieved if the subject were treated with a combination of lenalidomide and dexamethasone, comprising:
a) manufacturing daratumumab;
b) providing the HCP information that treatment with the combination therapy comprising daratumumab, lenalidomide and dexamethasone achieves the improved clinical efficacy endpoint; and
c) shipping daratumumab to the HCP or to an authorized distributor of daratumumab for the HCP to purchase daratumumab; thereby providing daratumumab to the HCP.

97) The method of claim 96, wherein the improved clinical efficacy endpoint is an increased likelihood of achieving a CR or better, an increased likelihood of achieving a VGPR or better, an increased likelihood of achieving a negative status for MRD, a reduced risk of progression of multiple myeloma or death, a prolonged progression-free survival (PFS), or an increased likelihood of achieving a 30-month rate of progression-free survival, or any combination thereof.

98) The method of claim 97, wherein the likelihood of achieving the CR or better is about 47% or higher, the likelihood of achieving the VGPR or better is about 79% or higher, the likelihood of achieving the negative status for MRD is about 24% or higher or the risk of progression of multiple myeloma or death is reduced by about 44%, or any combination thereof.

99) The method of claim 98, wherein the subject with newly diagnosed multiple myeloma is ineligible for HDC and ASCT.

100) The method of claim 99, wherein the combination therapy comprises about 16 mg/kg daratumumab, about 25 mg lenalidomide and between about 20 mg and about 40 mg dexamethasone.

101) The method of claim 100, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

102) The method of claim 101, wherein dexamethasone is administered as pre-medication on daratumumab administration days.

103) The method of claim 102, wherein daratumumab is administered intravenously, lenalidomide is administered orally and dexamethasone is administered intravenously or orally.

104) The method of claim 103, wherein lenalidomide, dexamethasone or both lenalidomide and dexamethasone are self-administered.

105) The method of claim 104, wherein information that the combination therapy comprising daratumumab, lenalidomide and dexamethasone achieves the improved clinical efficacy endpoint is provided on a daratumumab-containing drug product label.

106) The method of claim 105, wherein the daratumumab-containing drug product label includes information that a recommended dose of daratumumab is 16 mg/kg administered as an intravenous injection.

107) The method of claim 106, wherein the daratumumab-containing drug product label includes information that the recommended dosing schedule of daratumumab in combination with lenalidomide is once a week on weeks 1 to 8, once in two weeks on weeks 9-24 and thereafter once in four weeks.

108) The method of claim 107, wherein the daratumumab-containing drug product label includes information that the recommended dosing schedule of lenalidomide is 25 mg daily on days 1-21 of repeated 4 week cycles.

109) The method of claim 108, wherein the daratumumab-containing drug product label includes information that the recommended dosing schedule of dexamethasone is about 20 mg or about 40 mg per week.

110) The method of claim 109, wherein the daratumumab-containing drug product label includes data from an open-label, randomized active-controlled phase 3 study that compared treatment with daratumumab, lenalidomide and dexamethasone (DRd) to treatment with lenalidomide and dexamethasone (Rd) in subjects with newly diagnosed multiple myeloma who are ineligible for HDC and ASCT.

111) The method of claim 110, wherein the daratumumab-containing drug product label includes data that treatment with DRd resulted in about 44% reduction in the risk of multiple myeloma progression or death when compared to treatment with Rd.

112) The method of claim 111, wherein the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

113) The method of claim 112, wherein the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

114) The method of claim 113, wherein the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

115) The method of claim 114, wherein the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

116) The method of claim 115, wherein the daratumumab-containing drug product label includes data from a phase 3 active-controlled study that compared treatment with daratumumab, bortezomib and dexamethasone (DVd) to treatment with bortezomib and dexamethasone (Vd) in relapsed, refractory or relapsed and refractory multiple myeloma.

117) The method of claim 116, wherein the daratumumab-containing drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide bortezomib and dexamethasone.

118) The method of claim 117, wherein the daratumumab-containing drug product label includes information that side effects of daratumumab includes weakness, decreased appetite, bronchitis and lung infection.

119) The method of claim 118, wherein the daratumumab-containing drug product label includes information about approved indications, dosage and administrations, adverse reactions, drug product interactions, use in specific populations, clinical pharmacology, nonclinical toxicology, clinical studies and storage and handling of daratumumab, or any combination thereof.

120) The method of claim 119, wherein daratumumab is produced in a mammalian cell line.

121) The method of claim 120, wherein the mammalian cell line is a Chinese hamster ovary (CHO) cell line.

122) The method of claim 121, wherein the molecular weight of daratumumab is about 148 kDa.

123) The method of claim 99, wherein dexamethasone can be substituted for a dexamethasone equivalent, wherein the dexamethasone equivalent is methylprednisolone, prednisolone, prednisone or betamethasone, or any combination thereof.

124) A method of providing a treatment option for a HCP to treat a subject with newly diagnosed multiple myeloma with a combination therapy comprising daratumumab, lenalidomide and dexamethasone, wherein the treatment with the combination therapy comprising daratumumab, lenalidomide and dexamethasone achieves an improved clinical efficacy endpoint when compared to a clinical efficacy endpoint achieved if the subject were treated with a combination of lenalidomide and dexamethasone, comprising:
a) manufacturing daratumumab;
b) providing the HCP information that the combination therapy comprising daratumumab, lenalidomide and dexamethasone achieves the improved clinical efficacy endpoint; and
c) shipping daratumumab to the HCP or to an authorized distributor of daratumumab for the HCP to purchase daratumumab, thereby providing the treatment option for the HCP.

125) The method of claim 124, wherein the improved clinical efficacy endpoint is an increased likelihood of achieving a CR or better, an increased likelihood of achieving a VGPR or better, an increased likelihood of achieving a negative status for MRD, a reduced risk of progression of multiple myeloma or death, a prolonged progression-free survival (PFS), or an increased likelihood of achieving a 30-month rate of progression-free survival, or any combination thereof.

126) The method of claim 125, wherein the likelihood of achieving the CR or better is about 47% or higher, the likelihood of achieving the VGPR or better is about 79% or higher, the likelihood of achieving the negative status for MRD is about 24% or higher or the risk of progression of multiple myeloma or death is reduced by about 44%, or any combination thereof.

127) The method of claim 126, wherein the subject with newly diagnosed multiple myeloma is ineligible for HDC and ASCT.

128) The method of claim 127, wherein the combination therapy comprises about 16 mg/kg daratumumab, about 25 mg lenalidomide and between about 20 mg and about 40 mg dexamethasone.

129) The method of claim 128, wherein the combination therapy comprises about 16 mg/kg daratumumab administered once a week on weeks 1 to 8, once in two weeks on weeks 9-24 and thereafter once in four weeks, about 25 mg lenalidomide administered daily on days 1-21 of repeated 4-week cycles, and about 20 mg to about 40 mg dexamethasone administered per week.

130) The method of claim 129, wherein dexamethasone is administered as pre-medication on daratumumab administration days.

131) The method of claim 130, wherein daratumumab is administered intravenously, lenalidomide is administered orally and dexamethasone is administered intravenously or orally.

132) The method of claim 131, wherein lenalidomide, dexamethasone or both lenalidomide and dexamethasone are self-administered.

133) The method of claim 132, wherein information that the combination therapy comprising daratumumab, lenalidomide and dexamethasone achieves the improved clinical efficacy endpoint is provided on a daratumumab-containing drug product label.

134) The method of claim 133, wherein the daratumumab-containing drug product label includes information that a recommended dose of daratumumab is 16 mg/kg administered as an intravenous injection.

135) The method of claim 134, wherein the daratumumab-containing drug product label includes information that the recommended dosing schedule of daratumumab in combination with lenalidomide is once a week on weeks 1 to 8, once in two weeks on weeks 9-24 and thereafter once in four weeks.

136) The method of claim 135, wherein the daratumumab-containing drug product label includes information that the recommended dosing schedule of lenalidomide is 25 mg daily on days 1-21 of repeated 4 week cycles.

137) The method of claim 136, wherein the daratumumab-containing drug product label includes information that the recommended dosing schedule of dexamethasone is about 20 mg or about 40 mg per week.

138) The method of claim 137, wherein the daratumumab-containing drug product label includes data from an open-label, randomized active-controlled phase 3 study that compared treatment with daratumumab, lenalidomide and dexamethasone (DRd) to treatment with lenalidomide and dexamethasone (Rd) in subjects with newly diagnosed multiple myeloma who are ineligible for HDC and ASCT.

139) The method of claim 138, wherein the daratumumab-containing drug product label includes data that treatment with DRd resulted in about 44% reduction in the risk of multiple myeloma progression or death when compared to treatment with Rd.

140) The method of claim 139, wherein the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

141) The method of claim 140, wherein the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

142) The method of claim 141, wherein the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

143) The method of claim 142, wherein the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

144) The method of claim 143, wherein the daratumumab-containing drug product label includes data from a phase 3 active-controlled study that compared treatment with daratumumab, bortezomib and dexamethasone (DVd) to treatment with bortezomib and dexamethasone (Vd) in relapsed, refractory or relapsed and refractory multiple myeloma.

145) The method of claim 144, wherein the daratumumab-containing drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide bortezomib and dexamethasone.

146) The method of claim 145, wherein the daratumumab-containing drug product label includes information that side effects of daratumumab includes weakness, decreased appetite, bronchitis and lung infection.

147) The method of claim 146, wherein the daratumumab-containing drug product label includes information about approved indications, dosage and administrations, adverse reactions, drug product interactions, use in specific populations, clinical pharmacology, nonclinical toxicology, clinical studies and storage and handling of daratumumab, or any combination thereof.

148) The method of claim 147, wherein daratumumab is produced in a mammalian cell line.

149) The method of claim 148, wherein the mammalian cell line is a Chinese hamster ovary (CHO) cell line.

150) The method of claim 149, wherein the molecular weight of daratumumab is about 148 kDa.

151) The method of claim 127, wherein dexamethasone can be substituted for a dexamethasone equivalent, wherein the dexamethasone equivalent is methylprednisolone, prednisolone, prednisone or betamethasone, or any combination thereof.

152) A combination therapy comprising daratumumab, lenalidomide and dexamethasone for providing a treatment of a subject with newly diagnosed multiple myeloma, wherein the treatment achieves an improved clinical efficacy endpoint when compared to a clinical efficacy endpoint achieved if the subject were treated with a combination of lenalidomide and dexamethasone.

153) The combination therapy of claim 152, wherein the subject with multiple myeloma is ineligible for HDC and ASCT.

154) The combination therapy of claim 153, comprising about 16 mg/kg daratumumab, about 25 mg lenalidomide and about 20 mg to about 40 mg dexamethasone.

155) The combination therapy of claim 154, wherein the treatment of the subject with newly diagnosed multiple myeloma comprises administering to the subject about 16 mg/kg daratumumab once a week, once in two weeks or once in four weeks, about 25 mg lenalidomide daily and about 20 mg to about 40 mg dexamethasone per week.

156) The combination therapy of claim 155, wherein the treatment of the subject with newly diagnosed multiple myeloma comprises administering to the subject about 16 mg/kg daratumumab once a week on weeks 1-8, once in two weeks on weeks 9-24 and once in four weeks thereafter, about 25 mg lenalidomide once daily on days 1-21 of repeated 4 week cycles and about 20 mg or about 40 mg per week dexamethasone.

157) The combination therapy of claim 156, which is demonstrated to increase a likelihood of achieving a VGPR or better in subjects with newly diagnosed multiple myeloma.

158) The combination therapy of claim 157, wherein the likelihood of achieving the VGPR or better is about 79% or more.

159) The combination therapy of claim 158, which is demonstrated to increase a likelihood of achieving a negative status for MRD in subjects with newly diagnosed multiple myeloma.

160) The combination therapy of claim 159, wherein the likelihood of achieving the negative status for MRD is about 24% or more.

161) The combination therapy of claim 160, which is demonstrated to increase a likelihood of achieving a CR or better in subjects with newly diagnosed multiple myeloma.

162) The combination therapy of claim 161, wherein the likelihood of achieving the CR or better is about 47% or more.

163) The combination therapy of claim 162, which is demonstrated to reduce a risk of progression of multiple myeloma or death in subjects with newly diagnosed multiple myeloma.

164) The combination therapy of claim 163, wherein the risk of progression of multiple myeloma or death is reduced by about 44%.

165) The combination therapy of claim 164, wherein the combination therapy is promoted by a manufacturer of daratumumab for treatment of newly diagnosed multiple myeloma on a daratumumab-containing drug product label.

166) The combination therapy of claim 165, wherein the daratumumab-containing drug product label includes data from an open-label, randomized active-controlled phase 3 study that compared treatment with daratumumab in combination with lenalidomide and dexamethasone (DRd) to treatment with lenalidomide and dexamethasone (Rd) in patients with newly diagnosed multiple myeloma.

167) The combination therapy of claim 166, wherein the daratumumab-containing drug product label includes data that treatment with DRd resulted in about 44% reduction in the risk of multiple myeloma progression or death when compared to treatment with Rd.

168) The combination therapy of claim 167, wherein the daratumumab-containing drug product label includes data that treatment with DRd resulted in about 79.3% of subjects achieving VGPR or better, about 24% of subjects achieving a negative status for MRD, or about 47.6% of subjects achieving CR or better, or any combination thereof.

169) The combination therapy of any claim 168, wherein the daratumumab-containing drug product label includes a Kaplan-Meier curve of progression-free survival (PFS) comparing subjects having newly diagnosed multiple myeloma treated with DRd to subjects having newly diagnosed multiple myeloma treated with Rd.

170) The combination therapy of claim 169, wherein the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

171) The combination therapy of claim 170, wherein the daratumumab-containing drug product label includes data from a phase 3 active-controlled study that compared treatment with daratumumab, lenalidomide and dexamethasone (DRd) to treatment with lenalidomide and dexamethasone (Rd) in relapsed, refractory or relapsed and refractory multiple myeloma.

172) The combination therapy of claim 171, wherein the daratumumab-containing drug product label includes data from a phase 3 active-controlled study that compared treatment with daratumumab, bortezomib and dexamethasone (DVd) to treatment with bortezomib and dexamethasone (Vd) in relapsed, refractory or relapsed and refractory multiple myeloma.

173) The combination therapy of claim 172, wherein the daratumumab-containing drug product label includes drug product interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

174) The combination therapy of claim 173, wherein the daratumumab-containing drug product label includes information that side effects of daratumumab includes weakness, decreased appetite, bronchitis and lung infection.

175) The combination therapy of claim 174, wherein the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

176) The combination therapy of claim 154, wherein dexamethasone can be substituted for a dexamethasone equivalent, wherein the dexamethasone equivalent is methylprednisolone, prednisolone, prednisone or betamethasone, or any combination thereof.

177) A drug product comprising daratumumab that is provided in a package comprising one or more single-dose vials comprising daratumumab and a drug product label that includes information that treatment of a subject with newly diagnosed multiple myeloma with a combination therapy comprising daratumumab, lenalidomide and dexamethasone achieves an improved clinical efficacy endpoint when compared to a clinical efficacy endpoint achieved if the subject were administered a combination of lenalidomide and dexamethasone.

178) The drug product of claim 177, wherein the one or more single-dose vials comprises 100 mg daratumumab in 5 mL of solution or 400 mg daratumumab in 20 mL of solution.

179) The drug product of claim 178, wherein the one or more single-dose vials comprising 100 mg daratumumab in 5 mL of solution and the one or more single-dose vials comprising 400 mg daratumumab in 20 mL of solution further comprises glacial acetic acid, mannitol, polysorbate 20, sodium acetate trihydrate and sodium chloride.

180) The drug product of claim 179, wherein the one or more single-dose vials comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and the one or more single-dose vials comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

181) The drug product of claim 177, wherein the drug product label includes information that a recommended dosing schedule of daratumumab is 16 mg/kg once a week on weeks 1 to 8, once in two weeks on weeks 9-24 and thereafter once in four weeks, the recommended dosing schedule of lenalidomide is 25 mg daily on days 1-21 of repeated 4-week cycles, and the recommended dosing schedule of dexamethasone is 20 mg per week or 40 mg per week.

182) The drug product of claim 181, wherein the drug product label includes data from an open-label, randomized active-controlled phase 3 study that compared treatment with daratumumab in combination with lenalidomide and dexamethasone (DRd) to treatment with lenalidomide and dexamethasone (Rd) in patients with newly diagnosed multiple myeloma.

183) The drug product of claim 182, wherein the drug product label includes data that treatment with DRd resulted in about 44% reduction in the risk of multiple myeloma progression or death when compared to treatment with Rd.

184) The drug product of claim 183, wherein the drug product label includes data that treatment with DRd resulted in about 79.3% of subjects achieving VGPR or better, about 24% of subjects achieving a negative status for MRD, or about 47.6% of subjects achieving CR or better, or any combination thereof.

185) The drug product of claim 184, wherein the drug product label includes a Kaplan-Meier curve of progression-free survival (PFS) comparing subjects having newly diagnosed multiple myeloma treated with DRd to subjects having newly diagnosed multiple myeloma treated with Rd.

186) The drug product of claim 185, wherein the drug product label includes data from a phase 3 active-controlled study that compared treatment with daratumumab, bortezomib, melphalan and prednisone (D-VMP) to treatment with bortezomib, melphalan and prednisone (VMP).

187) The drug product of claim 186, wherein the drug product label includes data from a phase 3 active-controlled study that compared treatment with daratumumab in combination with lenalidomide and dexamethasone (DRd) to treatment with lenalidomide and dexamethasone (Rd) in relapsed, refractory or relapsed and refractory multiple myeloma.

188) The drug product of claim 187, wherein the drug product label includes data from a phase 3 active-controlled study that compared treatment with daratumumab in combination with bortezomib and dexamethasone (DVd) to treatment with bortezomib and dexamethasone (Vd) in relapsed, refractory or relapsed and refractory multiple myeloma.

189) The drug product of claim 188, wherein the drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

190) The drug product of claim 189, wherein the drug product label includes information that side effects of daratumumab includes feeling weak, decreased appetite, bronchitis and lung infection.

191) The drug product of claim 190, wherein the drug product label includes information about approved indications, dosage and administrations, adverse reactions, drug interactions, use in specific populations, clinical pharmacology, nonclinical toxicology, clinical studies and storage and handling of daratumumab, or any combination thereof.

192) A method of selling a drug product comprising daratumumab, comprising:
a) manufacturing daratumumab;
b) promoting that a combination therapy comprising daratumumab, lenalidomide and dexamethasone achieves an improved clinical efficacy endpoint when administered to a subject with newly diagnosed multiple myeloma, when compared to a clinical efficacy endpoint achieved if the subject were administered a combination of lenalidomide and dexamethasone, wherein performing the steps a) and b) results in a HCP to purchase the drug product; thereby selling the drug product.

193) The method of claim 192, wherein promoting comprises including data from an open-label, randomized active-controlled phase 3 study that compared treatment with daratumumab in combination with lenalidomide and dexamethasone (DRd) to treatment with lenalidomide and dexamethasone (Rd) in patients with newly diagnosed multiple myeloma on the drug product label.

194) The method of claim 193, wherein the drug product label further includes data that treatment with DRd resulted in about 44% reduction in the risk of multiple myeloma progression or death when compared to treatment with Rd.

195) The method of claim 194, wherein the drug product label further includes a Kaplan-Meier curve of progression-free survival (PFS) comparing subjects having newly diagnosed multiple myeloma treated with DRd to subjects having newly diagnosed multiple myeloma treated with Rd.

196) The method of claim 195, wherein daratumumab is produced in a mammalian cell line.

197) The method of claim 196, wherein the mammalian cell line is a Chinese hamster ovary (CHO) cell line.

198) The method of claim 197, wherein the molecular weight of daratumumab is about 148 kDa.

199) A method of selling a drug product comprising daratumumab, comprising
i) manufacturing daratumumab;
ii) selling the drug product, wherein the drug product label includes an indication for treating a subject with newly diagnosed multiple myeloma with a combination of daratumumab, lenalidomide and dexamethasone.

200) The method of claim 299, wherein daratumumab is produced in a mammalian cell line.

201) The method of claim 200, wherein the mammalian cell line is a Chinese hamster ovary (CHO) cell line.

202) The method of claim 201, wherein the molecular weight of daratumumab is about 148 kDa.
